(19) United States
(12) Patent Application Publication
(10) Pub. No.: US 2013/0017265 A1
(43) Pub. Date: Jan. 17, 2013

(54) PARTICLES FOR MULTIPLE AGENT DELIVERY

(71) Applicants: Omid C. Farokhzad, Chestnut Hill, MA (US); Nagesh Kolishetti, Athens, GA (US); Shanta Dhar, Cambridge, MA (US); Stephen J. Lippard, Cambridge, MA (US); Robert S. Langer, Newton, MA (US); Pedro M. Valencia, Cambridge, MA (US)

(72) Inventors: Omid C. Farokhzad, Chestnut Hill, MA (US); Nagesh Kolishetti, Athens, GA (US); Shanta Dhar, Cambridge, MA (US); Stephen J. Lippard, Cambridge, MA (US); Robert S. Langer, Newton, MA (US); Pedro M. Valencia, Cambridge, MA (US)

(73) Assignees: MASSACHUSETTS INSTITUTE OF TECHNOLOGY, Cambridge, MA (US); THE BRIGHAM AND WOMEN'S HOSPITAL, INC., Boston, MA (US)

(21) Appl. No.: 13/515,668

(22) PCT Filed: Dec. 16, 2010
(86) PCT No.: PCT/US10/60814

§ 371 Date: Sep. 14, 2012

Related U.S. Application Data
(60) Provisional application No. 61/287,188, filed on Dec. 16, 2009.

(51) Int. Cl. A61K 31/80; A61K 31/787; C08G 63/91; A61P 37/04; A61P 35/00; A61K 9/14; A61K 31/785

(52) U.S. Cl. 424/490; 424/78.17; 525/415; 525/450

(57) ABSTRACT

Delivery compositions are provided that include two or more active agents, wherein at least one active agent is conjugated to a polymer. The delivery compositions allow for controlled release of multiple active agents, including active agents with varying solubility, charge, and/or molecular weight.

### CLAIM OF PRIORITY

This application claims priority to U.S. Patent Application Ser. No. 61/287,188, filed on Dec. 16, 2009, the entire contents of which are incorporated herein by reference.

### STATEMENT AS TO FEDERALLY SPONSORED RESEARCH

This invention was made with Government support under Grant Nos. EB003647 and U54-CA119349, awarded by the National Institutes of Health. The Government has certain rights in the invention.

### TECHNICAL FIELD

This invention relates to particulate drug delivery compositions that include two or more active agents.

### BACKGROUND

Anti-cancer agents directed to an individual molecular target frequently show limited efficacies, poor safety, and poor resistance profiles. The genomic revolution and the advances in systems biology have identified potential synergistic therapies that may be concurrently utilized for more effective treatment of cancers. However, targeted delivery of multiple therapeutic agents has proven difficult. While it is possible to non-covalently encapsulate drugs within polymeric nanoparticles and release them in a regulated manner, the use of encapsulation strategies for combination drug delivery can result in batch-to-batch variability in encapsulation and release of multiple drugs especially when using drugs with varying solubility, charge, and molecular weight.

### SUMMARY

The development of nanotechnologies for effective delivery of multiple drugs or drug candidates to specific diseased cells and tissues, e.g., to cancer cells, e.g., to cancer cells in specific organs or tissues, in a tempospatially regulated manner can potentially overcome the therapeutic challenges faced to date. The present invention provides methods and compositions for controlling the formation of delivery compositions and release of active agents from the drug delivery compositions, e.g., particles. In one embodiment, the present invention provides methods for preparing drug delivery compositions, e.g., particles, that include active agents conjugated to polymers, e.g., polymers having pendant functional groups. One advantage of the present invention is that by engineering and blending distinct drug-functionalized and ligand-functionalized polymers, particles capable of delivering two, three, or more drugs can be reproducibly engineered and characterized. Additionally, these methods allow for characteristics of drug release and pharmacokinetics to be tuned, e.g. separately for each type of agent, e.g., regardless of the characteristics of the active agents, e.g., solubility, charge, molecular weight, half-life, and/or biodistribution profiles. Further, by targeting drug-loaded particles to specific tissues or cells, e.g., cancer cells, synergistic drug effects can be achieved that can alter the biodistribution of active agents. This can translate to better efficacy and tolerability, making active agents suitable for potential clinical development.

In one aspect, the invention features particles that include a polymer matrix containing a first active agent and a second active agent, wherein the polymer matrix is configured to provide release kinetics of each of the first and second active agents such that less than 50% of each of the first and second active agents is released within the first two hours (e.g., the first 4, 6, 8, 10, 12, 15, 20, 25, or 30 hours) of suspending the particles in a neutral aqueous solution, e.g., a buffered saline solution (e.g., PBS), at about 37° C. In some embodiments, the polymer matrix further includes one or more additional active agents, and can also be configured to provide controlled, e.g., similar, release kinetics of one or more of the additional active agents. In some embodiments, the matrix is configured to provide release kinetics of each of the first and second active agents independently such that less than 50% of the first active agent is released within the first two hours (e.g., the first 4, 6, 8, 10, 12, 15, 20, 25, or 30 hours) and less than 50% of the second active agent is released within the first two hours (e.g., the first 4, 6, 8, 10, 12, 15, 20, 25, or 30 hours).

In another aspect, the invention features particles that include a polymer matrix containing a first active agent and a second active agent, wherein the polymer matrix is configured such that each of the first and second active agents has a half life in the circulation of a subject of at least 30 minutes, one hour, or two hours (e.g., at least 4, 6, 8, 10, 12, 15, 20, 25, or 30 hours). In some embodiments, the polymer matrix further includes one or more additional active agents, and can also be configured to provide similar release kinetics of one or more of the additional active agents. In some embodiments, the polymer matrix is configured such that the first active agent has a half life in the circulation of the subject of at least 30 minutes, one hour, or two hours (e.g., at least 4, 6, 8, 10, 12, 15, 20, 25, 30 hours) when within the particles, and the second agent independently has a half life in the circulation of a subject of at least 30 minutes, one hour, or two hours (e.g., at least 4, 6, 8, 10, 12, 15, 20, 25, 30 hours) when within the particles. In some embodiments, the polymer matrix is configured such that the first active agent and/or the second active agent has a half life in the circulation of the subject that is at least 30 minutes, one hour, or two hours (e.g., 4, 6, 8, 10, 12, 15, 20, 25, 30 hours) longer than the active agent in the absence of the polymer matrix.

In some embodiments of the above particles, the first and/or second active agent is conjugated to a biodegradable polymer, e.g., a biodegradable polymer having pendant functional groups. Any additional active agents can also be conjugated to a biodegradable polymer, e.g., a biodegradable polymer having pendant functional groups.

In some aspects, the invention features particles having multiple active agents, wherein the particles include (a) a hydrophobic polymeric core containing (i) a first active agent conjugated to a biodegradable polymer, e.g., a biodegradable polymer having pendant functional groups, and; (ii) a second active agent; and (b) a hydrophilic layer that is surface-exposed. In some embodiments, the biodegradable polymer is a block copolymer having a first end that is relatively hydrophobic and a second end that is relatively hydrophilic, wherein the hydrophobic core includes the first end of the block copolymer conjugated to the first active agent, and wherein the hydrophilic layer includes the second end of the block copolymer. In some embodiments, the hydrophilic layer includes a plurality of amphiphilic block copolymers, each having a relatively hydrophobic end that interacts with the hydrophobic polymeric core and a relatively hydrophilic end that is surface-exposed. In some embodiments, the biodegradable polymer and conjugated active agent are included completely in the hydrophobic core. In other embodiments, the biodegradable polymer is amphiphilic and present in both the hydrophobic core and the hydrophilic layer. Of course, these embodiments are not mutually exclusive.

In one aspect, the invention features a particle having multiple active agents, wherein the particle includes (a) a hydrophobic polymeric core containing (i) a first active agent (e.g., a hydrophilic active agent or a hydrophobic active agent) conjugated to a biodegradable polymer, e.g., a biodegradable polymer having pendant functional groups and; (ii) a second active agent (e.g., a hydrophilic active agent or a hydrophobic active agent); and (b) an amphiphilic shell that includes a plurality of amphiphilic block copolymers having a hydrophobic end that interacts with the hydrophobic polymeric core and a hydrophilic end that is surface-exposed. In some embodiments, the amphiphilic shell further includes a plurality of targeting block copolymers, each having a hydrophobic end that interacts with the hydrophobic polymeric core and a hydrophilic end that is conjugated to a targeting agent. In some embodiments, the second active agent is also conjugated to a biodegradable polymer, e.g., a biodegradable polymer having pendant functional groups. In some embodiments, the second active agent is a hydrophobic active agent that is not conjugated to a biodegradable polymer.

In some embodiments of the above particles, the targeting agent comprises an aptamer, nucleic acid, nucleic acid ligand, polypeptide, protein ligand, small molecule, growth factor, hormone, cytokine, interleukin, antibody, antibody fragment, integrin, fibronectin receptor, carbohydrate, p-glycoprotein receptor, peptide, peptidomimetic, hydrocarbon, small modular immunopharmaceutical, cell binding sequence, Affibody, Nanobody, Adnectin, Domain Antibody, or an Avimer, or any combination thereof. In some embodiments, the targeting agent is a peptide comprising fewer than 8 amino acids.

In some of the embodiments of the above particles, the targeting agent binds to the Prostate Specific Membrane Antigen (PSMA). In one non-limiting example, the targeting agent can be an A10 aptamer.

In some embodiments of the above particles, the biodegradable polymer includes a polylactic acid, polycaprolactone, polyglycolic acid, polyanhydride, or poly(lactide-co-glycolic acid) or a derivative of any thereof. In some embodiments of the above particles, the biodegradable polymer having pendant functional groups includes a derivative of a polylactic acid, polycaprolactone, polyglycolic acid, polyanhydride, or poly(lactide-co-glycolic acid). In some embodiments, the biodegradable polymer is a copolymer, e.g., a block copolymer.

Exemplary hydrophilic active agents include cisplatin, carboplatin, mitaplatin, oxaliplatin, methyl jasmonate, dichloroacetate, or irinotecan, and derivatives or prodrugs thereof. Exemplary hydrophobic active agents include paclitaxel, docetaxel, gefitinib, tubacin, betulinic acid, resveratrol, alpha-tocopheryl succinate, or combretastatin, and derivatives or prodrugs thereof.

In some embodiments, the first active agent and second active agent are independently selected from a biomolecule, bioactive agent, small molecule, drug, prodrug, drug derivative, protein, peptide, vaccine, adjuvant, imaging agent (e.g., a fluorescent moiety) or polynucleotide.

Any of the active agents described herein can be included in the particles with any other active agent described herein or other active agents. In some embodiments, the first and second active agents are, respectively, paclitaxel or docetaxel and gefitinib; gefitinib and paclitaxel or docetaxel; oxaliplatin (or oxaliplatin prodrug) and irinotecan; irinotecan and oxaliplatin (or oxaliplatin prodrug); paclitaxel and tubacin; tubacin and placlitaxel; lonidamine, dichloroacetate, alpha-tocopheryl succinate, betulinic acid, or resveratrol and Pt(IV) hexanoate; Pt(IV) hexanoate and lonidamine, dichloroacetate, alpha-tocopheryl succinate, betulinic acid, or resveratrol; alpha-tocopheryl succinate or methyl jasmonate and docetaxel; or docetaxel and alpha-tocopheryl succinate or methyl jasmonate. These particles can further include a third active agent, e.g., combretestatin.

The particles disclosed herein can include additional active agents, e.g., three, four, five, six, or more active agents. In some embodiments, one or more of the additional active agents (e.g., a third active agent) is conjugated to a biodegradable polymer, e.g., a biodegradable polymer having pendant functional groups.

In some embodiments, the particles disclosed herein have an average diameter of about 100 μm or less, e.g., about 10 μm or less, about 1000 nm or less, about 800 nm or less, about 600 nm or less, about 500 nm or less, about 400 nm or less, about 300 nm or less, about 250 nm or less, about 200 nm or less, about 100 nm or less, about 80 nm or less, about 60 nm or less, about 50 nm or less, or about 40 nm or less. In some embodiments, a plurality of the particles disclosed herein is provided. The plurality of particles can have an average characteristic dimension of about 100 μm or less, e.g., about 10 μm or less, about 1000 nm or less, about 800 nm or less, about 600 nm or less, about 500 nm or less, about 400 nm or less, about 300 nm or less, about 250 nm or less, about 200 nm or less, about 100 nm or less, about 80 nm or less, about 60 nm or less, about 50 nm or less, or about 40 nm or less. In some embodiments, the plurality of particles has a polydispersity index of 0.8 or less, e.g., 0.6 or less, 0.4 or less, 0.2 or less, or 0.1 or less.

In some embodiments, the invention features pharmaceutical compositions that include any of the above particles and, optionally, one or more pharmaceutically acceptable carriers and/or diluents. The pharmaceutical compositions can be formulated, e.g., for intravenous, intra-arterial, oral, transdermal, transmucosal, intraperitoneal, intracranial, intraocular, epidural, intrathecal, topical, enema, injection, pulmonary route or infusion delivery.

In some aspects, the invention features methods of preparing nanoparticles having multiple active agents. The methods can include dissolving a first active agent (e.g., a hydrophilic active agent or a hydrophobic active agent) conjugated to a biodegradable polymer, e.g., a biodegradable polymer having pendant functional groups in a volatile, water-miscible organic solvent to form a first solution; dissolving a second active agent (e.g., a hydrophilic active agent or a hydrophobic active agent) in a volatile, water-miscible organic solvent to form a second solution; dissolving a plurality of amphiphilic block copolymers in a water-miscible organic solvent to form a third solution; and combining the first, second, and third solutions such that a nanoparticle is formed having a hydrophobic polymeric core surrounded by the amphiphilic block copolymers.

In some aspects, the invention features methods of formulating particles that include at least two active agents. The methods can include providing a first and second active agent; conjugating the first active agent, or a prodrug or derivative thereof, to a biodegradable polymer, e.g., a biodegradable polymer having pendant functional groups; and preparing a particle comprising the conjugated first active agent and the second active agent. In some embodiments, the methods further include conjugating the second active agent, or a prodrug or derivative thereof, to a biodegradable polymer, e.g., a biodegradable polymer having pendant functional groups. In some embodiments, the first and second active agents are incompatible for formation of a particle in the absence of the biodegradable polymer. In such a case, conjugating the first active agent and/or the second active agent to a biodegradable polymer can impart compatibility of the first and second active agents for formation of a particle. In some embodiments of the above methods, the particle can be formed by precipitation, emulsion, or emulsion and solvent evaporation. In some embodiments, the particle is formed using a microfluidics apparatus.

In some aspects, the invention features methods of delivering multiple active agents to a biological target within a subject. The methods can include obtaining a pharmaceutical composition comprising a plurality of particles disclosed herein that include a targeting agent, wherein the targeting agent binds specifically to the biological target; and administering to the subject the pharmaceutical composition in an amount effective to deliver the active agents in the particles to the biological target. In some embodiments, the targeting agent specifically binds to a tumor cell or tumor vasculature.

In other aspects, the invention features methods of delivering multiple active agents to a subject. The methods can include obtaining a pharmaceutical composition comprising a plurality of particles disclosed herein; and administering to the subject the pharmaceutical composition in an amount effective to deliver the active agents.

In additional aspects, the invention features methods of treating a disorder, e.g., a cancer or other disorder disclosed herein, in a subject in need thereof, the method comprising administering to the subject an effective amount of a particle described above, wherein the first and second active agents are selected to treat the disorder.

In some aspects, the invention features methods of tempospatially controlling administration of two or more active agents to a subject. The methods can include providing a particle comprising a polymer matrix comprising a first active agent and a second active agent, wherein the polymer matrix is configured to provide desired tempospatial release kinetics of each of the first and second active agents; and administering the particle to a subject such that the first and second active agents are released from the particle with the desired tempospatial release kinetics. In some embodiments, the desired tempospatial release kinetics comprise release of the drugs at a desired location, e.g., an organ, tissue, or cell, or a tumor or tumor vasculature. In some embodiments, the desired tempospatial release kinetics comprise release of the first and second active agent such that a desired dosage of each of the first and second active agents is provided to the subject. In some embodiments, one or both of the first and second active agents is conjugated to a biodegradable polymer, e.g., a biodegradable polymer having pendant functional groups.

In any of the above methods of administration, the particle or plurality of particles can be delivered intravenously, intra-arterially, orally, transdermally, transmucosally, intraperitoneally, intracranially, intraocularly, epidurally, intrathecally, topically, by enema, by injection, by pulmonary route or by infusion.

As used herein, a “hydrophilic active agent” is one that has a solubility in water at 20° C. and one atmosphere of pressure of 50 mg/L or greater, e.g., 100 mg/L or greater, 200 mg/L or greater, 500 mg/L or greater, 1.0 g/L or greater, 2.0 g/L or greater, or 5.0 g/L or greater.

As used herein, a “hydrophobic active agent” is one that has a solubility in water at 20° C. and one atmosphere of pressure of less than 50 mg/L, e.g., less than 20 mg/L, less than 10 mg/L, less than 5.0 mg/L, less than 2.0 mg/L, or less than 1.0 mg/L.

In some cases, the hydrophilicity of two or more active agents can be measured relative to each other, i.e., a first active agent can be more hydrophilic than a second active agent. For instance, the first active agent can have a greater solubility in water than the second active agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a schematic of hydroxyl functionalized polylactides (PLA-OH) conjugated with drugs (circle, square or triangle) and various polylactide-polyethylene glycol (PLA-PEG) and PLA-PEG conjugated to a targeting ligand.

FIG. 1B is a schematic of a targeted nanoparticle that includes hydroxyl functionalized polylactides conjugated with drugs (circle, square or triangle) and the effect of the combination therapy on cancer cell death.

FIG. 2 depicts ¹H-NMR characterization of the conversion of functionalized polylactides. PLA-OH is created by deprotection of benzyl groups from PLA-OBn and was visualized by a decrease in the intensity of phenyl rings at 7.3 ppm. The presence of a platinum prodrug in PLA-Pt was visualized by the appearance of amino protons at 6.3 ppm after conjugation with the platinum (IV) monosuccinate prodrug.

FIG. 3 is a line graph showing the in vitro toxicity of PLA-OH nanoparticles (PLA-OH-NP).

FIGS. 4A-4B are schematics that depict the formation of exemplary nanoparticles containing poly(lactic-co-glycolic acid)-polyethylene glycol-COOH (PLGA-PEG-COOH) with PLA-OH conjugated to Pt(IV)-monosuccinate (poly-Pt) and docetaxel. The nanoparticles can be prepared by nanoprecipitation (4A) or by microfluidics methods (4B).

FIG. 5A is a histogram that shows the sizes of Poly-Pt-Doce-NP as determined by dynamic light scattering.

FIG. 5B is a transmission electron micrograph of Poly-Pt-Doce-NP.

FIGS. 6A-6B are cyclic voltammograms of c,c,t[Pt(NH₃)Cl₂(OH)(succinate)] in DMF-0.1 M TBAPF₆ (6A) and Poly-Pt(IV) in DMF-0.1 M TBAPF₆ (6B) at various scan rates.

FIGS. 6C-6D are plots of the respective reduction peak potential maxima of in the voltammograms vs. scan rate of the voltammograms of FIGS. 6A and 6B, respectively.

FIGS. 7A-7B show in vitro release of platinum (7A) and docetaxel (7B) from dual drug Poly-Pt-Doce-NP.

FIGS. 8A-8B are line graphs depicting cytotoxicity of Pt(IV) monosuccinate (monosuccinate), cisplatin, PolyPt-NP, and PolyPt-NP-Apt on LNCaP (8A) and PC3 (8B) cells.

FIG. 9 is a set of micrographs depicting uptake by LNCaP cells of PolyPt-NP and targeted PolyPt-NP-Apt. DIC, differential interference contrast; FITC, fluorescein isothiocyanate; EEA1, mouse monoclonal antibody.

FIG. 10A is a schematic of formation of a platinum-DNA adduct.

FIG. 10B is a set of micrographs depicting detection of a Pt-GG adduct in LNCaP cells following treatment of the cells with PolyPt-Doce-NP-Apt or PolyPt-NP-Apt.

FIG. 11 is a schematic of exemplary syntheses of PLA-OH, PLA-COOH, and PLA-drug. For conjugation to PLA-OH and PLA-COOH, other chemistries can be utilized (e.g., T-NH₂).

FIG. 12 is a schematic showing formation of PLA-Pt from PLA-OH.

FIG. 13 is a schematic of construction of a dual-drug nanoparticle comprising PLA-Pt and docetaxel, optionally conjugated to a targeting ligand (A10 aptamer).

FIG. 14 is a schematic of a combinatorial semi-automated process for development of targeted polymeric nanoparticles. A microfluidic system with multiple inlets is used for introduction of distinct drug-functionalized polymers, ligand-functionalized polymers, free polymers, and free drugs to mix the desired precursors. The mixing ratio is governed by input flow that is automatically controlled by syringe pumps that interface with a PC. The resulting polymeric and drug precursors are precipitated in an aqueous solution through flow focusing in microfluidic channels. Each combination of precursor flow rate results in a distinct nanoparticle formulation, which is automatically collected in a 96 well plate for further purification and analysis.

FIG. 15A is a schematic of the synthesis of PLA-COOH.

FIG. 15B depicts ¹H-NMR characterization of PLA-COOH.

FIG. 16A is a schematic of the synthesis of PLA-Lonidamine.

FIG. 16B depicts ¹H-NMR spectral stack of PLA-Lonidamine (PLA-Loni).

FIG. 17A is a schematic of the synthesis of PLA-dichloroacetate (PLA-DCA).

FIG. 17B depicts ¹H-NMR spectral stack of PLA-DCA obtained by the reaction of dichloroacetic anhydride with PLA-OH in the presence of catalytic amount of base N,N-Diisopropylethylamine (DIPEA).

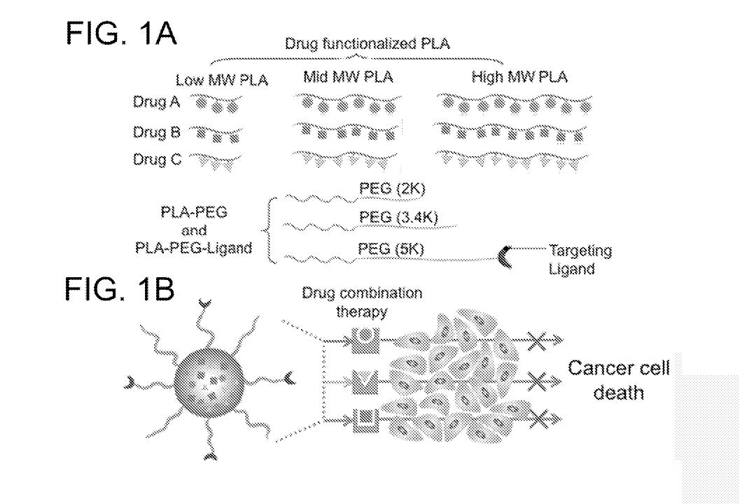

### DETAILED DESCRIPTION

The present application provides drug delivery compositions that include two or more active agents, at least one of which is conjugated to a biodegradable polymer, e.g., a biodegradable polymer having pendant functional groups. The delivery compositions allow for co-delivery of multiple drugs, e.g., with different characteristics, e.g., hydrophilic and hydrophobic drugs together with the ability to independently control the release parameters of each drug. Additionally, the delivery compositions can include a targeting agent for delivery of the systems to desired cellular targets. As one example, a PLA-functionalized dual drug delivery composition was developed by combining docetaxel inside a platinum-modified PLA polymer resulting in moderately high loading with particles of suitable size for delivering cisplatin and docetaxel simultaneously to prostate cancer cells.

### Delivery Compositions

In one aspect, the delivery compositions are particles with that include two or more active agents. Active agents can be conjugated to a biodegradable polymer, e.g., a biodegradable polymer having pendant functional groups. The particles can also include an amphiphilic shell surrounding the hydrophobic core that contains a plurality of amphiphilic molecules, e.g., amphiphilic lipids or amphiphilic copolymers. The particles can further include a targeting agent on the surface of the particle.

In addition, the particles, e.g., polymeric micro- and nanoparticles, can be produced such that they are biodegradable, such that they include materials already approved by FDA, and such that they result in a submicron size (e.g., 10 nm-1000 nm or other ranges, e.g., 25 nm-250 nm, e.g., 15 nm-50 nm, 10 nm-500 nm), or a micron-scale size. Nano-scale particles are considered herein to be up to 1000 nm at their largest cross-sectional dimension. Micron-scale particles are over 1.0 micron at their largest cross-sectional dimension (e.g., 1.0 micron up to 100 microns, or larger, e.g., 1.0 to 2.0 microns, 1.0 to 10.0 microns, 5 to 25 microns, and 25 to 50 microns), can also be made according to the methods described herein.

In some cases, the particle is a nanoparticle, i.e., the particle has a characteristic dimension of less than 1 micrometer, where the characteristic dimension is the largest cross-sectional dimension of a particle. For example, the particle can have a characteristic dimension of less than about 500 nm, less than about 400 nm, less than about 250 nm, less than about 200 nm, less than about 150 nm, less than about 100 nm, less than about 50 nm, less than about 30 nm, less than about 10 nm, less than about 3 nm, or less than about 1 nm in some cases.

In some cases, a population of particles can be present. Various embodiments of the present invention are directed to such populations of particles. For instance, in some embodiments, the population of particles can have an average characteristic dimension of less than about 500 nm, less than about 400 nm, less than about 250 nm, less than about 200 nm, less than about 150 nm, less than about 100 nm, less than about 50 nm, less than about 30 nm, less than about 10 nm, less than about 3 nm, or less than about 1 nm in some cases. In some embodiments, the particles can each be substantially the same shape and/or size (“monodisperse”). For example, the particles can have a distribution of characteristic dimensions such that no more than about 5% or about 10% of the particles have a characteristic dimension greater than about 10% greater than the average characteristic dimension of the particles, and in some cases, such that no more than about 8%, about 5%, about 3%, about 1%, about 0.3%, about 0.1%, about 0.03%, or about 0.01% have a characteristic dimension greater than about 10% greater than the average characteristic dimension of the particles. In some cases, no more than about 5% of the particles have a characteristic dimension greater than about 5%, about 3%, about 1%, about 0.3%, about 0.1%, about 0.03%, or about 0.01% greater than the average characteristic dimension of the particles.

In some embodiments, the diameter of no more than 25% of the produced particles varies from the mean particle diameter by more than 150%, 100%, 75%, 50%, 25%, 20%, 10%, or 5% of the mean particle diameter. It is often desirable to produce a population of particles that is relatively uniform in terms of size, shape, and/or composition so that each particle has similar properties. For example, at least 80%, at least 90%, or at least 95% of the particles produced using the methods described herein can have a diameter or greatest dimension that falls within 5%, 10%, or 20% of the average diameter or greatest dimension. In some embodiments, a population of particles can be heterogeneous with respect to size, shape, and/or composition. See, e.g., PCT publication WO 2007/150030, which is incorporated herein by reference in its entirety.

In some embodiments, the polydispersity index of a population of particles is 0.6 or less, e.g., 0.5 or less, 0.4 or less, 0.3 or less, 0.2 or less, 0.1 or less, or 0.05 or less.

In many embodiments, the particles are formulated for controlled release. Controlled release occurs when a natural or synthetic polymer is combined with an active agent in such a way that the drug is retained within the polymer system for subsequent release in a predetermined manner. Polymeric drug delivery compositions that are designed as particles can release the conjugated active agents through surface or bulk erosion, diffusion, and/or swelling followed by diffusion, in a time or condition dependent manner. The release of the active agent can be constant over a long or short period, it can be cyclic over a long or short period, or it can be triggered by the environment or other external events (see, e.g., Langer and Tirrell, 2004, Nature, 428:487-492). In general, controlled-release polymer systems can provide drug levels in a specific range over a longer period of time than other drug delivery methods, thus increasing the efficacy of the drug and maximizing patient compliance.

While PLA and PLGA can be used to non-covalently encapsulate drugs and release them in a regulated manner, the use of strategies for combination drug delivery can result in batch-to-batch variability in release of multiple drugs, especially when using drugs with varying characteristics, e.g., solubility, charge, molecular weight, half-life, biodistribution profiles. In contrast, the use of delivery compositions that include drug-polymer conjugates as described herein offers more control over the load and release of drugs especially when delivering drugs with varying solubility, charge, and molecular weight.

Without wishing to be bound by theory, the particle parameters, e.g., size, charge, etc., can alter the delivery (e.g., loss of payload, drug efflux, aggregations, delivery to desired location, etc.) of the active agents from the particles. In some cases, larger particles tend to lose their payload more quickly than smaller particles and/or a drug efflux may be more rapid from smaller particles than larger particles. Smaller particles, in some cases, can be more likely to aggregate than larger particles. The size of the particle may affect the distribution of the particles throughout the body. For example, larger particles injected into a bloodstream may be more likely to be lodged in small vessels than smaller particles. In some instances, larger particles may be less likely to cross biological barriers (e.g., capillary walls) than smaller particles. The size of the particles used in a delivery composition can be selected based on the application, and will be readily known to those of ordinary skill in the art. For example, particles of smaller size (e.g., <200 nm) can be selected if systematic delivery of the particles throughout a patient's bloodstream is desired. As another example, particles of larger size (e.g., >200 nm) can be selected if sequestering of the particles by a patient's reticuloendothelial system upon injection is desired (e.g., sequestering of the particles in the liver, spleen, etc.). The desired length of time of delivery can also be considered when selecting particle size. For example, smaller particles tend to circulate in the blood stream for longer periods of time than larger particles.

In some embodiments, the particles are designed to substantially accumulate at the site of a specific target, e.g. a tumor. In some embodiments, this may be due, at least in part, the presence of a targeting moiety associated with the particle, as described herein. In some embodiments, this may be due, at least in part, due to an enhanced permeability and retention (EPR) effect, which allows for particles to accumulate specifically at a tumor site. The EPR effect will be known to those of ordinary skill in the art and refers to the property by which certain sizes of material (e.g., particles) tend to accumulate in tumor tissue much more than they do in normal tissues.

Polymers

In some embodiments, the delivery compositions comprise one or more polymeric base components (e.g., a polymer). A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer. In some cases, additional moieties can also be present in the polymer, for example targeting moieties such as those described herein.

If more than one type of repeat unit is present within the polymer, then the polymer is said to be a “copolymer.” It is to be understood that in any embodiment employing a polymer, the polymer being employed can be a copolymer in some cases. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In some embodiments, a polymer is amphiphilic, i.e., having a hydrophilic portion and a hydrophobic portion, or a relatively hydrophilic portion and a relatively hydrophobic portion. A hydrophilic polymer is one that generally attracts water and a hydrophobic polymer is one that generally repels water. A hydrophilic or a hydrophobic polymer can be identified, for example, by preparing a sample of the polymer and measuring its contact angle with water (typically, a hydrophilic polymer will have a contact angle of less than about 50°, while a hydrophobic polymer will have a contact angle of greater than about 50°). In some cases, the hydrophilicity of two or more polymers can be measured relative to each other, i.e., a first polymer can be more or less hydrophilic than a second polymer. For instance, the first polymer can have a smaller contact angle than the second polymer. In embodiments containing more than two polymers, the polymers can be ranked in order by comparing their solubility parameters.

In one set of embodiments, the polymer base component (e.g., polymer) can be biocompatible, i.e., a polymer that does not typically induce an adverse response when inserted or injected into a living subject, for example, without significant inflammation and/or acute rejection of the polymer by the immune system, for instance, via a T-cell response. It will be recognized, of course, that “biocompatibility” is a relative term, and some degree of immune response is to be expected even for polymers that are highly compatible with living tissue. However, as used herein, “biocompatibility” refers to the lack of acute rejection of material by at least a portion of the immune system, i.e., a nonbiocompatible material implanted into a subject provokes an immune response in the subject that is severe enough such that the rejection of the material by the immune system cannot be adequately controlled, and often is of a degree such that the material must be removed from the subject. One simple test to determine biocompatibility is to expose a polymer to cells in vitro; biocompatible polymers are polymers that typically do not result in significant cell death at moderate concentrations, e.g., at concentrations of about 50 micrograms/10⁶ cells. For instance, a biocompatible polymer may cause less than about 20% cell death when exposed to cells such as fibroblasts or epithelial cells, even if phagocytosed or otherwise uptaken by such cells. Non-limiting examples of biocompatible polymers that can be useful in various embodiments of the present invention include polydioxanone (PDO), polyhydroxyalkanoate, polyhydroxybutyrate, poly(glycerol sebacate), polyglycolide, polylactide, polycaprolactone, polyanhydride or copolymers or derivatives including these and/or other polymers.

In certain embodiments, the biocompatible polymer is biodegradable, i.e., the polymer is able to degrade, chemically and/or biologically, within a physiological environment, such as within the body. For instance, the polymer can be one that hydrolyzes spontaneously upon exposure to water (e.g., within a subject), the polymer can degrade upon exposure to heat (e.g., at temperatures of about 37° C.). Degradation of a polymer can occur at varying rates, depending on the polymer or copolymer used. For example, the half-life of the polymer (the time at which 50% of the polymer is degraded into monomers and/or other nonpolymeric moieties) can be on the order of days, weeks, months, or years, depending on the polymer. The polymers can be biologically degraded, e.g., by enzymatic activity or cellular machinery, in some cases, for example, through exposure to a lysozyme (e.g., having relatively low pH). In some cases, the polymers can be broken down into monomers and/or other nonpolymeric moieties that cells can either reuse or dispose of without significant toxic effect on the cells (for example, polylactide can be hydrolyzed to form lactic acid, polyglycolide can be hydrolyzed to form glycolic acid, etc.). Examples of biodegradable polymers include, but are not limited to, poly(lactide) (or poly(lactic acid)), poly(glycolide) (or poly(glycolic acid)), poly(orthoesters), poly(caprolactones), polylysine, poly(ethylene imine), poly(acrylic acid), poly(urethanes), poly(anhydrides), poly(esters), poly(trimethylene carbonate), poly(ethyleneimine), poly(acrylic acid), poly(urethane), poly(beta amino esters) or the like, and copolymers or derivatives of these and/or other polymers, for example, poly(lactide-co-glycolide) (PLGA).

In another set of embodiments, a polymer of the present invention can be able to control immunogenicity, for example a poly(alkylene glycol) (also known as poly(alkylene oxide)), such as poly(propylene glycol), or poly(ethylene oxide), also known as poly(ethylene glycol) (“PEG”), having the formula —(CH₂—CH₂—O)ₙ—, where n is any positive integer. In some embodiments, branched PEGs can be used (see, e.g., Veronese et al., 2008, BioDrugs, 22:315-329; Hamidi et al., 2006, Drug Deliv., 13:399-409). The poly(ethylene glycol) units can be present within the polymeric base component in any suitable form. For instance, the polymeric base component can be a block copolymer where one of the blocks is poly(ethylene glycol). A polymer comprising poly(ethylene glycol) repeat units is also referred to as a “PEGylated” polymer. Such polymers can control inflammation and/or immunogenicity (i.e., the ability to provoke an immune response), due to the presence of the poly(ethylene glycol) groups. PEGylation can also be used, in some cases, to decrease charge interaction between a polymer and a biological moiety, e.g., by creating a hydrophilic layer on the surface of the polymer, which can shield the polymer from interacting with the biological moiety. For example, PEGylation can be used to create particles which comprise an interior which is more hydrophobic than the exterior of the particles. In some cases, the addition of poly(ethylene glycol) repeat units can increase plasma halflife of the polymeric conjugate, for instance, by decreasing the uptake of the polymer by the phagocytic system while decreasing transfection/uptake efficiency by cells. Those of ordinary skill in the art will know of methods and techniques for PEGylating a polymer, for example, by using EDC (1-ethyl-3-(3-dimethylaminopropyl)carbodiimide hydrochloride) and NHS (N-hydroxysuccinimide) to react a polymer to a PEG group terminating in an amine, for example, by ring opening polymerization techniques (ROMP), or the like. In addition, certain embodiments of the invention are directed towards copolymers containing poly(ester-ether)s, e.g., polymers having repeat units joined by ester bonds (e.g., R—C(O)—O—R′ bonds) and ether bonds (e.g., R—O—R′ bonds).

The polymers described herein can be prepared with pendant functional groups, i.e., functional groups present along a length of the polymer, e.g., a portion of the polymer, for conjugation to active agents. In some embodiments, the polymer has approximately one functional group (e.g., conjugated functional group) for every 1, 2, 3, 4, 5, 6, 7, 8, 9, 10, 12, 15, or 20 monomer units of the polymer. The functional groups can be restricted to one portion of the polymer, e.g., in a block copolymer. In some embodiments, the functional groups are hydroxyl, carboxyl, amine, amide, carbamate, maleimide, thiol, halide, azide, proparzyl, allyl, etc. Additionally, the functional groups can be joined to the polymer by a linker.

Various methods are known to conjugate a heterofunctional linker to an active agent using covalent bonds (such as including σ-bonding, π-bonding, metal to non-metal bonding, agnostic interactions, disulfide bonds, and three-center two-electron bonds). In one example, a bond, e.g., crosslinking, can be achieved by forming an amide bond between carboxyl (or maleimide) and a primary amine by using 1-ethyl-3-[3-dimethylaminopropyl]carbodiimide hydrochloride/N-hydroxysuccinimide (EDC/NHS) or benzotriazole-1-yl-oxy-tris-pyrrolidino-phosphonium hexafluorophosphate/N-hydroxybenzotriazole (pyBOP/HOBt). The reaction can tolerate both aqueous and organic solvents (such as, but not limited to, dichloromethane, acetonitrile, chloroform, tetrahydrofuran, acetone, formamide, dimethylformamide, pyridines, dioxane, or dimethysulfoxide).

In another example, binding, e.g., crosslinking is formed between maleimide and sulfhydryl (thiol) groups in both aqueous and organic solvents. A reduction cleavable crosslinking can be achieved between sulfhydryl (thiol) group, through the pyridylthiol group, 3-nitro-2-pyridylthio (Npys) group, and Boc-S-tert-butylmercapto (StBu) group. The reaction can tolerate both aqueous and organic solvents (such as, but not limited to, dichloromethane, acetonitrile, chloroform, tetrahydrofuran, acetone, formamide, dimethylformamide, pyridines, dioxane, or dimethysulfoxide).

Methods of making functionalized polymers with pendant functional groups are known in the art. For example, methods of making and functionalized polylactides, polyglycolides, polyesteramides are described in Gerhardt et al., 2006, Biomacromolecules, 7:1735-42. Synthesis of functionalized dilactones and use in preparation of polyesters with hydroxyl functional groups, poly(lactic acid-co-hydroxymethyl glycolic acid) and poly(lactic acid-co-glycolic acid-co-hydroxymethyl glycolic acid) is described in Leemhuis et al., 2007, Biomacromolecules, 8:2943-49; Leemhuis et al., 2003, Eur. J. Org. Chem., 3344-49; and Leemhuis et al., 2006, Macromolecules, 39:3500-08. Hydroxymethyl and succinylated PLA polymers are described in Noga et al., 2008, Biomacromolecules, 9:2056-62. Poly hexyl-substituted lactides are disclosed in Trimaille et al., 2005, Chimia 59:348-352; and Trimaille et al., 2007, J. Biomed. Mater. Res., 80A:55-65. Jing and Hillmyer, 2008, J. Am. Chem. Soc., 130:13826-27 describe PLA with functional bicyclic esters. Poly(α-hydroxy acid)s with pendant carboxyl groups are disclosed in Kimura et al., 1988, Macromolecules, 21:3338-40. Additionally, biodegradable esters can be derived from amino acids, as described in Kolitz et al., 2009, Macromolecules, DOI: 10.1021/ma900464g; Cohen-Arazi et al., 2008, 41:7259-63. In some embodiments, the polymers can be functionalized by “click” methods, as described in Jiang et al., 2008, Macromolecules, 41:1937-44.

Additional methods of synthesis and conjugation of polymers are disclosed in US 20080248126, which is incorporated herein by reference in its entirety.

The length of the polymers, e.g., having pendant functional groups, which are conjugated to active agents can be varied to provide desired parameters. Typically, increasing the length of the polymer decreases the rate of release of the active agent. In some embodiments, the polymers have a molecular weight of about 2000 g/mol before conjugation to active agents, e.g., about 3000 g/mol, about 4000 g/mol, about 5,000 g/mol, about 10,000 g/mol, about 20,000 g/mol, about 50,000 g/mol, or about 100,000 g/mol.

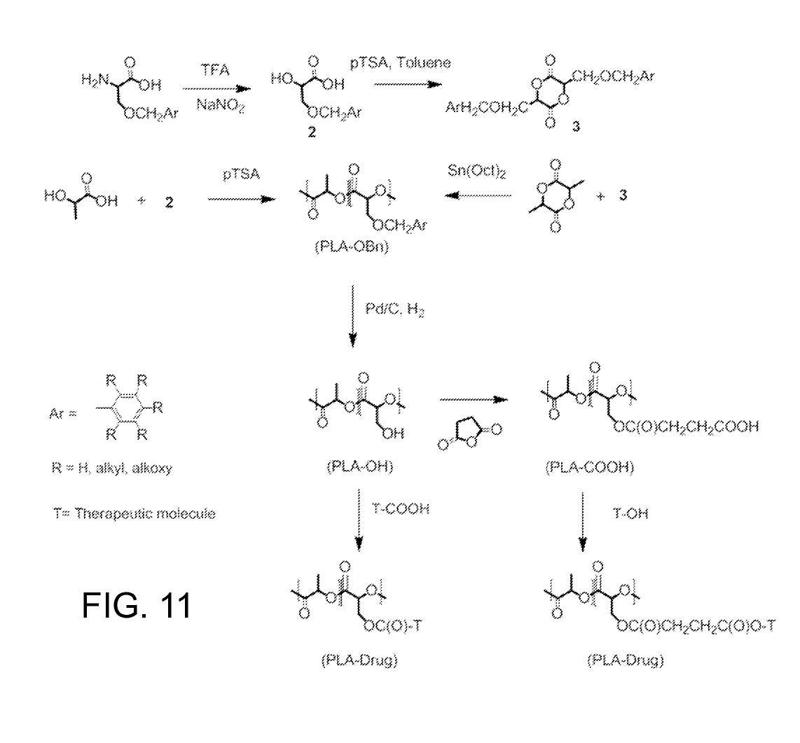

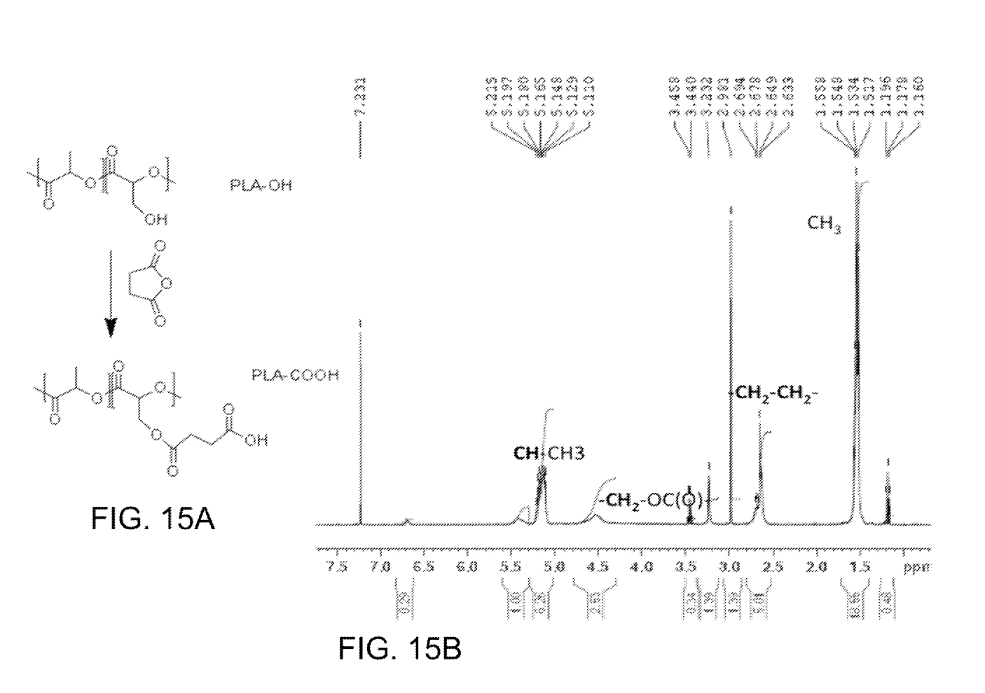

The incorporation of reactive functional groups capable of forming bonds allows for conjugation of distinct drugs and subsequent hydrolysis of this bond in physiological conditions resulting in drug release in a controlled manner. FIG. 11 shows an exemplary approach for the development of biodegradable polylactide derivatives with pendant hydroxyl groups. Briefly, an amine is converted to hydroxyl via diazotization reaction using sodium nitrite in presence of an acid. The resultant monomer 2 can be directly used for condensation polymerization in conjunction with lactic acid to give a polylactide copolymer, Poly-OBn. The same polymer can be made using ring opening polymerization (ROP) of the cyclic lactide monomer 3, which is made via dehydration reaction of the α-hydroxyl acid under very dilute reaction conditions in toluene with para-toluene sulfonic acid. For the synthesis of high molecular weight polymers, the ROP approach is favored. The benzyl protecting group prevents side reactions of the hydroxyl group during the polymerization. The hydroxyl functionalized biodegradable polylactide (PLA-OH) can then be synthesized via benzyl deprotection using Pd/C catalyst. For the synthesis of a carboxyl functionalized polylactide, PLA-OH can be treated with an anhydride, e.g., succinic anhydride or itaconic anhydride (see FIG. 15A).

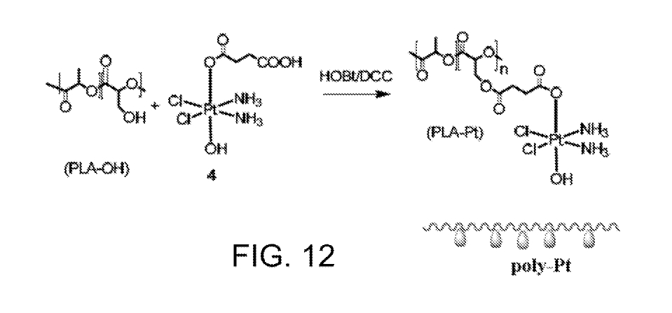

FIG. 12 shows an exemplary strategy for conjugation of a cisplatin prodrug. Platinum monosuccinate 4, a prodrug of cisplatin, is conjugated with the PLA-OH polymer using DCC/HOBT coupling to generate final PLA-Pt (FIG. 12). Similar strategies can be used for the conjugation of other drug molecules.

Active Agents

The particles include two or more active agents, at least one of which is conjugated to a polymer, e.g., a polymer having pendant functional groups. The active agents selected can be suitable for use in a wide variety of applications (e.g., therapeutic, imaging, and diagnostic applications) and include proteins, peptides, sugars, lipids, steroids, DNA, RNA, small molecule drugs, and prodrugs of any of agents described herein. As used herein, a prodrug is a pharmacological substance that is metabolized in vivo into a pharmaceutically active form. In some cases, the prodrug is pharmaceutically inactive or significantly less active than the pharmaceutically active form.

In some embodiments, the active agent is a small molecule drug. The term “small molecule” is art-recognized and refers to a composition which has a molecular weight of less than about 2000 g/mole, less than about 1500 g/mole, less than about 1000 g/mole, less than about 800 g/mole, less than about 700 g/mole, less than about 600 g/mole, less than about 500 g/mole, less than about 400 g/mole, less than about 300 g/mole, less than about 200 g/mole, less than about 100 g/mole, or less. Those of ordinary skill in the art will be able to determine if a small molecule drug is suitable to be functionalized with a polymer, e.g., a polymer having pendant functional groups.

In some embodiments, an active agent is either hydrophilic or hydrophobic. A hydrophilic or a hydrophobic polymer can be identified, for example, by measuring the solubility of the active agent in water. In some cases, the hydrophilicity of two or more active agents can be measured relative to each other, i.e., a first active agent can be more hydrophilic than a second active agent. For instance, the first active agent can have a greater solubility in water than the second active agent. In embodiments containing more than two active agents, the active agents can be ranked in order by comparing their solubility parameters.

In some embodiments, the combination of active agents is synergistic for the treatment of a disorder. Extensive investigations of the molecular basis of drug actions and responses have yielded a substantial amount of information on experimentally determined drug-mediated molecular interaction profiles and regulatory activities of many drugs and compounds (see, e.g., Drews, 2000, Science, 287:1960-64; Imming et al., 2007, Nature Rev. Drug Discov. 5:821-834; Robertson, 2005, Biochemistry, 44:5561-71; Zybarth et al., 2006, Curr. Drug Targets, 7:387-395; Wishart et al., 2006, Nucleic Acids Res., 34:D668-D672; Ya, et al., 2006, Appl. Bioinformatics, 5:131-139; Liu et al., 2007, Nucleic Acids Res., 35:D198-D201; and Ji et al., 2003, Drug Discov. Today, 8:526-529). The molecular interaction profile of a drug describes its interactions with individual biomolecules, pathways or processes attributable to its pharmacodynamic, toxicological, pharmacokinetic, and combination effects. Molecular interaction profiles can be used to guide the development of combinations of active agents.

Suitable, non-limiting examples of active agents that can be used include 5-Fluorouracil (5-FU): an anti-metabolite drug commonly used in cancer treatment. Typical dosing begins with intravenous treatment at 400 mg/m² (i.e., per square meter of calculated body surface area) over 15 minutes as a bolus, then an ambulatory pump delivers 2,400 mg/m² as a continuous infusion over 46 hours. Suitable chemotherapeutic drugs can be divided into the following classes: alkylating agents, antimetabolites, anthracyclines, plant alkaloids, topoisomerase inhibitors, monoclonal antibodies, and other anti-tumor agents. In addition to the chemotherapeutic drugs described above, namely doxorubicin, paclitaxel, other suitable chemotherapy drugs include tyrosine kinase inhibitor imatinib mesylate (Gleevec® or Glivec®), cisplatin, carboplatin, oxaliplatin, mechloethamine, cyclophosphamide, chlorambucil, azathioprine, mercaptopurine, pyrimidine, vincristine, vinblastine, vinorelbine, vindesine, podophyllotoxin (L01CB), etoposide, docetaxel, topoisomerase inhibitors (L01CB and L01XX) irinotecan, topotecan, amsacrine, etoposide, etoposide phosphate, teniposide, dactinomycin, lonidamine, and monoclonal antibodies, such as trastuzumab (Herceptin®), cetuximab, bevacizumab and rituximab (Rituxan®), among others. Additional exemplary active agents include PARP inhibitors, survivin inhibitors, estradiol, and dichloroacetate.

Other examples of active agents include, but are not limited to, antimicrobial agents, analgesics, antinflammatory agents, counterirritants, coagulation modifying agents, diuretics, sympathomimetics, metabolic modulators, anorexics, antacids and other gastrointestinal agents; antiparasitics, antidepressants, antihypertensives, anticholinergics, stimulants, antihormones, central and respiratory stimulants, drug antagonists, lipid-regulating agents, uricosurics, cardiac glycosides, electrolytes, ergot and derivatives thereof, expectorants, hypnotics and sedatives, antidiabetic agents, dopaminergic agents, antiemetics, muscle relaxants, para-sympathomimetics, anticonvulsants, antihistamines, beta-blockers, purgatives, antiarrhythmics, contrast materials, radiopharmaceuticals, antiallergic agents, tranquilizers, vasodilators, antiviral agents, and antineoplastic or cytostatic agents or other agents with anticancer properties, or a combination thereof. Other suitable active agents include contraceptives and vitamins as well as micro- and macronutrients. Still other examples include antiinfectives such as antibiotics and antiviral agents; analgesics and analgesic combinations; anorexics; antihelminthics; antiarthritics; antiasthmatic agents; anticonvulsants; antidepressants; antidiuretic agents; antidiarrleals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins.

Exemplary metabolic modulators include lonidamine, dichloroacetate, alpha-tocopheryl succinate, methyl jasmonate, betulinic acid, and resveratrol. In some embodiments, the particles include an anticancer agent and a metabolic modulator.

In certain embodiments, the particles can include lovastatin, a cholesterol lowering and heart disease active agent, which can be included within the nanoparticles described herein. In another aspect, a suitable active agent included in core of the particle is Phenyloin, an anticonvulsant agent (marketed as Dilantin®) in the USA and as Epanutin® in the UK by Pfizer, Inc). Antibiotics can be incorporated into the particle, such as vancomycin, which is frequently used to treat infections, including those due to methicillin resistant staph aureus (MRSA). The particle optionally includes cyclosporin, a lipophilic drug that is an immunosuppressant agent, widely used post-allogeneic organ transplant to reduce the activity of the patient's immune system and the risk of organ rejection (marketed by Novartis under the brand names Sandimmune®, the original formulation, and Neoral® for the newer microemulsion formulation). Particles comprising cyclosporine can be used in topical emulsions for treating keratoconjunctivitis sicca, as well. In this regard, particles with multifunctional surface domains incorporating such drugs can be designed to deliver equivalent dosages of the various drugs directly to the cancer cells, thus potentially minimizing the amount delivered generally to the patient and minimizing collateral damage to other tissues.

In certain specific aspects, the particles of the present disclosure include one or more of: non-steroidal anti-inflammatory agents (NSAIDs), analgesics, COX-I and II inhibitors, and the like. For example, indomethacin is a suitable NSAID suitable for incorporation into a multiphase nano-component of the disclosure.

Other active agents in the form of therapeutic agents are described in WO 2008/124632, which is incorporated herein by reference in its entirety.

Non-limiting examples of hydrophilic drugs which can be functionalized with an auxiliary compatibilizing moiety includes cisplatin, carboplatin, mitaplatin, oxaliplatin, pyriplatin, Pt(IV) hexanoate, irinotecan, methyl jasmonate, dexamethasone phosphate, nicardipine hydrochloride, methylsalicylic acid, dichloroacetate, nitroglycerine, hydrophilic serotonin 5-HT₃ receptor antagonists (e.g., ondansetron, granisetron), aminotetralins (e.g., S(−)-2-(N-propyl-N-2-thienylethylamine)-5-hydroxytetralin), anthracyclines, etc. In some embodiments, the drug or drug precursor can comprise an inorganic or organometallic compound, for example, a platinum compound (as described herein), a ruthenium compound (e.g., trans-[RuCl₂(DMSO)₄], trans-[RuCl₂(imidazole)₂] etc.), cobalt compounds, copper compounds, iron compounds, etc.

In some embodiments, an inhibitor of nucleic acid repair is formulated in combination with a DNA-damaging agent, e.g., a platinum compound.

As an example, a cisplatin prodrug (platinum monosuccinate) was functionalized to a PLA having pendant hydroxyl groups (see Example 1). Additionally, docetaxel has been combined with PLA-Pt (drug load w/w %: docetaxel 3%; Pt 5%) and controlled release of cisplatin and docetaxel from nanoparticles was demonstrated over several days.

Similar approaches can be used for conjugation of other active agents. For example, for the development of polylactide with pendant oxaliplatin, the oxaliplatin prodrug can be synthesized with carboxyl groups at the axial position, which will be coupled to the PLA-OH. Polylactide with paclitaxel pendant groups was prepared by generating carboxyl group containing polylactide by treating PLA-OH with succinic anhydride, and this compound was coupled directly with hydroxyl groups of paclitaxel. In the case of tubacin-functionalized polymers, the same carboxyl group containing polylactides can be conjugated to the hydroxyl groups of tubacin.

In one aspect, the present invention provides compositions and methods that enable multiple active agents with varying chemical properties to be administered to patients, e.g., simultaneously in a safe, effective, and controlled manner. Combining multiple active agents into a single delivery composition also allows for targeting of the active agents to specific cellular targets, e.g., tumor cells. Indeed, the treatment efficacy of many traditional combination therapies (e.g., cancer treatments that use two or more drugs) is often limited because the dose-limiting toxicities (DLTs) of the individual drugs are lower when the two drugs are administered in combination than when they are administered individually. In such cases, the dose of each drug needs to be reduced in the combination therapy, thereby reducing the individual drug contributions to overall treatment efficacy. In addition, this hampers the opportunities for identifying novel synergisms. The present invention solves this problem by using an active agent conjugated to a biodegradable polymer as one or more of the combination therapeutics. Because these conjugates deliver their drugs in a targeted manner, they have higher dose-limiting toxicities than the drugs themselves. By using a conjugate as one or more of the combination therapeutics one can therefore increase the dose of one or more of the drugs in the combination. In one embodiment, two or more conjugates that each carry different drugs are administered in combination. In one embodiment, a conjugate is administered with one or more non-conjugated drugs. In any of these embodiments it is to be understood that one can increase the dose of just one or several drugs in the combination (e.g., one or both drugs in a combination of two drugs). It is also to be understood that one can increase the dose of a drug which is conjugated and/or the dose of a drug which is non-conjugated.

The methods and compositions of the present invention are in no way limited to specific drugs, specific drug combinations, or specific diseases, but certain combinations disclosed herein can provide beneficial and/or synergistic results.

For example, and without limitation, certain agents with known synergies can be combined into a single delivery composition. For example, paclitaxel or docetaxel with gefitinib has been shown to have a strong synergistic effect in breast cancer MCF7/ADR cells; oxaloplatin and irinotecan have a synergistic anticancer effect in AZ-521 and NUGC-4 cells; and paclitaxel and tubacin synergistically enhance tubulin acetylation. Additionally, combretastatin or another agent that blocks neovascularization can be incorporated into the delivery compositions, including delivery compositions that include targeting agents specific for PSMA. Other combinations that can be incorporated can be found, e.g., in Jia et al., 2009, Nat. Rev. Drug. Discov., 8:111-128, and include DL-cycloserine and epigallocatechin gallate; paclitaxel and NU6140; gefitinib and taxane; gefitinib and PD98059; AZT and non-nucleoside HIV-1 reverse transcriptase inhibitors; aplidin and cytarabine; gefitinib and ST1926; sildenafil and iloprost; dexmedetomidine and ST-91; mycophenolate mofetil and mizoribine; paclitaxel and discodermolide; ampicillin and daptomycin; candesartan-cilexetil and ramipril; diazoxide and dibutyryl-cGMP; propofol and sevoflurane; ampicillin and imipenem; artemisinin and curcumin; doxorubicin and trabectedin; and azithromycin and imipenem. Jia et al., Nat. Rev. Drug. Discov., 8:111-128, is incorporated herein by reference in its entirety

For example, and without limitation, certain metastatic breast cancers are currently treated with a combination of cyclophosphamide, methotrexate and fluorouracil (CMF) or a combination of cyclophosphamide, doxorubicin and fluorouracil (CAF). Thus, in one embodiment, two or three of the above agents in these combination therapies could be administered in a single particle.

Bladder, head and neck and endometrial cancers could similarly be treated by administering two or more of the individual drugs in M-VAC (methotrexate, vinblastin, adriamycin, cisplatin) or CMV (cisplatin, methotrexate, vinblastin) in a single particle.

One of ordinary skill will recognize variations on these embodiments for other traditional combination therapies (e.g., without limitation, any of those described in “Combination Cancer Therapy: Modulators and Potentiators”, Schwartz, Ed., Humana Press, 2004; “Combination Therapy of AIDS”, Ed. by DeClerq et al., Birkhauser, 2004; etc.).

Targeting Agents

In certain embodiments the inventive conjugates can be modified to include targeting agents that will direct an inventive conjugate to a particular cell type, collection of cells, or tissue. Preferably, the targeting agents are associated with the surface of the particles. A variety of suitable targeting agents are known in the art (Cotten et al., Methods Enzym. 217:618, 1993; Torchilin, Eur. J. Pharm. Sci. 11:881, 2000; Garnett, Adv. Drug Deliv. Rev. 53:171, 2001). For example, any of a number of different materials which bind to antigens on the surfaces of target cells can be employed. Antibodies to target cell surface antigens will generally exhibit the necessary specificity for the target. In addition to antibodies, suitable immunoreactive fragments can also be employed, such as the Fab, Fab′, or F(ab′)2 fragments. Many antibody fragments suitable for use in forming the targeting mechanism are already available in the art. Similarly, ligands for any receptors on the surface of the target cells can suitably be employed as targeting agent. These include any small molecule or biomolecule, natural or synthetic, which binds specifically to a cell surface receptor, protein or glycoprotein found at the surface of the desired target cell.

There are other targeting agents, such as nucleic acid ligands, such as aptamers, which are small oligonucleotides that specifically bind to certain target molecules and are potential candidates to target proteins over-expressed in cancer cells, such as prostate cancer cells. A nucleic acid ligand is a nucleic acid that can be used to bind to a specific molecule. For example, pegaptanib is a pegylated anti-VEGF aptamer, a single stranded nucleic acid that binds with high specificity to a particular target. Although the pegaptanib aptamer was originally approved by FDA in 2004 to treat age-related macular degeneration (AMD) disease, it has the potential to treat prostate cancer because it binds specifically to VEGF165, a protein recognized as the key inducer of tumor angiogenesis. Latil et al., Int. J. Cancer, 89, 167-171 (2000) suggests that VEGF expression could be used as a prognostic marker in early-stage tumors. Specific aptamers include, for example, Aptamer O-7 which binds to osteoblasts; A10 RNA aptamer, which binds to prostate cancer cells; aptamer TTA1, which binds to breast cancer cells; and the extended A9 RNA aptamer (Javier et al., Bioconjug. Chem. 2008 Jun. 18; 19(6):1309-1312). See also, Wilson et al., U.S. Published Patent Application No. 20090105172. In general, aptamers are stable in a wide range of pH (˜4-9), physiological conditions, and solvents. Aptamers are known to be less immunogenic than antibodies and can penetrate a tumor more easily because of size. The shape of aptamer binding sites, which includes grooves and clefts, provide highly specific characteristics and drug-like capabilities. Active targeting, however, requires that the RNA aptamers discriminate cancer cells from normal cells.

Other exemplary targeting agents include peptides, such as CLT1 and CLT2, which bind to fibrin-fibronectin complexes in blood clots. Various peptides are well known in the art for binding to cells in the brain, kidneys, lungs, skin, pancreas, intestine, uterus, adrenal gland, and prostate, including those described in Pasqualini et al., Mol. Psychiatry, 1:421-2 (1996) and Rajotte et al., J. Clin. Invest., 102:430-437 (1998), for example.

In one aspect of the invention, there can be two or more distinct targeting agents bound to the surface of a particle. A primary target can be an immune system cell, such as a leukocyte or T-cell, and a secondary target can be a malignant cancer cell(s) within a tumor, which is the target region. The targeting agent on the surface of particle binds to the primary target cell with high selectivity, while the second moiety has a general tumor targeting surface domain. Suitable moieties for binding with targets associated with an animal include those described herein. Thus, after delivery of the inventive multifunctional particles to the target tissue, the particles having tumor targeting moieties can bind with the secondary target (e.g., cancer) cells, once they detach from originally targeted cells. In certain aspects, a particle delivery composition is provided for active agent delivery that is long-circulating, highly selective, and enables the release of multiple drugs with complex release kinetics.

Other targeting agents include agents that specifically bind to biological targets such as a particular immune system cell (e.g., a T cell or B cell), a protein, an enzyme, or other circulating agent associated with a subject. The following provides are exemplary and non-limiting examples of suitable targeting moieties for use with the multifunctionalized particles described herein. Proteins, such as heat shock protein HSP70 for dendritic cells and folic acid to target cancer cells. Polysaccharides or sugars, such as silylic acid for targeting leucocytes, targeting toxins such as saporin, antibodies, including CD 2, CD 3, CD 28, T-cells, and other suitable antibodies are listed in a Table available on the internet on the World Wide Web at “researchd.com/rdicdabs/cdindex.htm,” incorporated herein by reference.

The term “binding,” as used herein, refers to the interaction between a corresponding pair of molecules or portions thereof that exhibit mutual affinity or binding capacity, typically due to specific or non specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones, or the like. The term “binding partner” refers to a molecule that can undergo binding with a particular molecule. “Specific binding” refers to binding by molecules, such as polynucleotides, antibodies, and other ligands, that are able to bind to or recognize a binding partner (or a limited number of binding partners) to a substantially higher degree than to other, similar biological entities. In one set of embodiments, the targeting moiety has a specificity (as measured via a disassociation constant) of less than about 1 micromolar, at least about 10 micromolar, or at least about 100 micromolar.

Non-limiting examples of targeting agents include a peptide, a protein, an enzyme, a nucleic acid, a fatty acid, a hormone, an antibody, a carbohydrate, a peptidoglycan, a glycopeptide, or the like. These and other targeting agents are discussed in detail below. In some cases, the biological targeting moiety can be relatively large, for example, for peptides, nucleic acids, or the like. For example, the biological moiety can have a molecular weight of at least about 1,000 Da, at least about 2,500 Da, at least about 3000 Da, at least about 4000 Da, or at least about 5,000 Da, etc. Relatively large targeting agents can be useful, in some cases, for differentiating between cells. For instance, in some cases, smaller targeting agents (e.g., less than about 1000 Da) may not have adequate specificity for certain targeting applications, such as targeting applications. In contrast, larger molecular weight targeting agents can offer a much higher targeting affinity and/or specificity. For example, a targeting agent can offer smaller dissociation constants, e.g., tighter binding. However, in other embodiments, the targeting agent can be relatively small, for example, having a molecular weight of less than about 1,000 Da or less than about 500 Da.

In one embodiment, the targeting agent includes a protein or a peptide. “Proteins” and “peptides” are well-known terms in the art, and are not precisely defined in the art in terms of the number of amino acids that each includes. As used herein, these terms are given their ordinary meaning in the art. Generally, peptides are amino acid sequences of less than about 100 amino acids in length, but can include sequences of up to 300 amino acids. Proteins generally are considered to be molecules of at least 100 amino acids. A protein can be, for example, a protein drug, an antibody, an antibody fragment, a recombinant antibody, a recombinant protein, an enzyme, or the like. In some cases, one or more of the amino acids of the protein or peptide can be modified in some instances, for example, by the addition of a chemical entity such as a carbohydrate group, a phosphate group, a farnesyl group, an isofarnesyl group, a fatty acid group, a linker for conjugation, functionalization, or other modification, etc.

Other examples of peptides or proteins include, but are not limited to, ankyrins, arrestins, bacterial membrane proteins, clathrin, connexins, dystrophin, endothelin receptor, spectrin, selectin, cytokines; chemokines; growth factors, insulin, erythropoietin (EPO), tumor necrosis factor (TNF), neuropeptides, neuropeptide Y, neurotensin, transforming growth factor alpha, transforming growth factor beta, interferon (IFN), and hormones, growth inhibitors, e.g., genistein, steroids etc; glycoproteins, e.g., ABC transporters, platelet glycoproteins, GPIb-IX complex, GPIIb-IIIa complex, vitronectin, thrombomodulin, CD4, CD55, CD58, CD59, CD44, CD168, lymphocyte function-associated antigen, intercellular adhesion molecule, vascular cell adhesion molecule, Thy-1, antiporters, CA-15-3 antigen, fibronectins, laminin, myelin-associated glycoprotein, GAP, and GAP43. Other examples include affibodies, nanobodies, Avimers, Adnectins, domain antibodies, and small modular immunopharmaceuticals (SMIP™) (Trubion Pharmaceuticals Inc., Seattle, Wash.).

As used herein, an “antibody” refers to a protein or glycoprotein consisting of one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well characterized fragments produced by digestion with various peptidases.

Non-limiting examples of antibodies and other suitable targeting agents include anti-cluster of differentiation antigen CD-1 through CD-166 and the ligands or counter receptors for these molecules; anti-cytokine antibodies, e.g., anti-IL-1 through anti-IL-18 and the receptors for these molecules; anti-immune receptor antibodies, antibodies against T cell receptors, major histocompatibility complexes I and II, B cell receptors, selectin killer inhibitory receptors, killer activating receptors, OX-40, MadCAM-1, Gly-CAM1, integrins, cadherens, sialoadherens, Fas, CTLA-4, Fc-gamma receptor, Fc-alpha receptors, Fc-epsilon receptors, Fc-mu receptors, and their ligands; anti-metalloproteinase antibodies, e.g., collagenase, MMP-1 through MMP-8, TIMP-1, TIMP-2; anti-cell lysis/proinflammatory molecules, e.g., perforin, complement components, prostanoids, nitrous oxide, thromboxanes; or anti-adhesion molecules, e.g., carcinoembryonic antigens, lamins, or fibronectins.

Other examples of targeting agents include cytokines or cytokine receptors, such as Interleukin-1 (IL-1), IL-2, IL-3, IL-4, IL-5, IL-6, IL-7, IL-8, IL-9, IL-10, IL11, IL-12, IL-13, IL-14, IL-15, IL-16, IL-17, IL-18, IL-1 receptor, IL-2 receptor, IL-3 receptor, IL-4 receptor, IL-5 receptor, IL-6 receptor, IL-7 receptor, IL-8 receptor, IL-9 receptor, IL-10 receptor, IL-11 receptor, IL-12 receptor, IL-13 receptor, IL-14 receptor, IL-15 receptor, IL-16 receptor, IL-17 receptor, IL-18 receptor, lymphokine inhibitory factor, macrophage colony stimulating factor, platelet derived growth factor, stem cell factor, tumor growth factor beta, tumor necrosis factor, lymphotoxin, Fas, granulocyte colony stimulating factor, granulocyte macrophage colony stimulating factor, interferon alpha, interferon beta, interferon gamma.

Still other examples of targeting agents include growth factors and protein hormones, for example, erythropoietin, angiogenin, hepatocyte growth factor, fibroblast growth factor, keratinocyte growth factor, nerve growth factor, tumor growth factor alpha, thrombopoietin, thyroid stimulating factor, thyroid releasing hormone, neurotrophin, epidermal growth factor, VEGF, ciliary neurotrophic factor, LDL, somatomedin, insulin growth factor, or insulin-like growth factor I and II.

Additional examples of targeting agents include chemokines, for example, ENA-78, ELC, GRO-alpha, GRO-beta, GRO-gamma, HRG, LIF, IP-10, MCP-1, MCP-2, MCP-3, MCP-4, MIP-1 alpha, MIP-1 beta, MIG, MDC, NT-3, NT-4, SCF, LIF, leptin, RANTES, lymphotactin, eotaxin-1, eotaxin-2, TARC, TECK, WAP-1, WAP-2, GCP-1, GCP-2, alpha-chemokine receptors such as CXCR1, CXCR2, CXCR3, CXCR4, CXCR5, CXCR6, CXCR7, or beta-chemokine receptors such as CCR1, CCR2, CCR3, CCR4, CCR5, CCR6, or CCR7.

In another embodiment, the targeting agent includes a nucleic acid. The term “nucleic acid,” or “oligonucleotide,” as used herein, refers to a polymer of nucleotides. As used herein, a “nucleotide” is given its ordinary meaning as used in the art, i.e., a molecule comprising a sugar moiety, a phosphate group, and a base (usually nitrogenous). Typically, the nucleotide comprises one or more bases connected to a sugar-phosphate backbone (a base connected only to a sugar moiety, without the phosphate group, is a “nucleoside”). The sugars within the nucleotide can be, for example, ribose sugars (a “ribonucleic acid,” or “RNA”), or deoxyribose sugars (a “deoxyribonucleic acid,” or “DNA”). In some cases, the polymer can comprise both ribose and deoxyribose sugars. Examples of bases include, but not limited to, the naturally-occurring bases (e.g., adenosine or “A,” thymidine or “T,” guanosine or “G,” cytidine or “C,” or uridine or “U”). In some cases, the polymer can also comprise nucleoside analogs (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, 2-thiocytidine, etc.), chemically or biologically modified bases (e.g., methylated bases), intercalated bases, modified sugars (e.g., 2′-fluororibose, 2′-azidoribose, 2′-azidoribose, 2′-O-methylribose, L-enantiomeric nucleosides arabinose, hexose, etc.), modified phosphate moieties (e.g., phosphorothioates or 5′-N-phosphoramidite linkages), and/or other naturally and non-naturally occurring bases substitutable into the polymer, including substituted and unsubstituted aromatic moieties. Other suitable base and/or polymer modifications are well-known to those of skill in the art. In some cases, the polynucleotide can include DNA, RNA, modified DNA, modified RNA, antisense oligonucleotides, expression plasmid systems, nucleotides, modified nucleotides, nucleosides, modified nucleosides, intact genes, or combinations thereof. Other examples of polynucleotides include interfering RNA, natural or unnatural siRNAs, shRNAs, microRNAs, ribozymes, DNA plasmids, antisense oligonucleotides, randomized oligonucleotides, or ribozymes.

Tumor targeted particles can be delivered into the tumor via the passive or active process. In the former, nanoparticles pass through leaky tumor capillary fenestrations into the tumor interstitium and cells by passive diffusion or convection. The latter involves drug delivery to a specific site based on molecular recognition. The most common approach conjugates targeting ligands to the nanoparticles. The targeting ligands enhance the interaction between nanoparticles and receptors at the target cell site, increasing local drug concentration. Many ligands have been successfully conjugated to the nanoparticles including antibodies, transferrin receptor, folate receptors, and wide range of biomolecules, as discussed above.

Examples of molecules targeting extracellular matrix (“ECM”) include glycosaminoglycan (“GAG”) and collagen. The outer surface of the particles that have a carboxy functional group can be linked to Pathogen-associated molecular patterns (PAMPs) that have a free amine terminus. The PAMPs target Toll-like Receptors (TLRs) on the surface of the cells or tissue, or signals the cells or tissue internally, thereby potentially increasing uptake. PAMPs conjugated to the particle surface or included in the particles can include: unmethylated CpG DNA (bacterial), double-stranded RNA (viral), lipopolysachamide (bacterial), peptidoglycan (bacterial), lipoarabinomannin (bacterial), zymosan (yeast), mycoplasmal lipoproteins such as MALP-2 (bacterial), flagellin (bacterial) poly(inosinic-cytidylic)acid (bacterial), lipoteichoic acid (bacterial) or imidazoquinolines (synthetic).

Lectins can also be used as targeting agents that can be covalently attached to the linkers of the new particles to target them to the mucin and mucosal cell layers. Such lectins can be isolated from Abrus precatroius, Agaricus bisporus, Anguilla anguilla, Arachis hypogaea, Pandeiraea simplicifolia, Bauhinia purpurea, Caragan arobrescens, Cicer arietinum, Codium fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

Several cell surface markers have been proposed as potential targets for tumor-homing therapeutics, including, for example, prostate-specific membrane antigen (PSMA), HER-2, HER-3, EGFR, and folate receptor. PSMA is a well established tumor marker, which is up-regulated in prostate cancer, particularly in advanced, hormone-independent, and metastatic disease (Ghosh and Heston, 2004, J. Cell. Biochem., 91:528-539). PSMA has been employed as a tumor marker for imaging of metastatic prostate cancer and as a target for experimental immunotherapeutic agents. PSMA is the molecular target of ProstaScint®, a monoclonal antibody-based imaging agent approved for diagnostic imaging of prostate cancer metastases. J591, a de-immunized monoclonal antibody that targets the external domain of PSMA, has been evaluated clinically as an agent for radioimmunotherapy and radioimmunoimaging. Radiolabeled J591 is reported to accurately target prostate cancer metastases in bone and soft tissue and to display anti-tumor activity. Interestingly, PSMA is differentially expressed at high levels on the neovasculature of most non-prostate solid tumors, including breast and lung cancers, and the clinical feasibility of PSMA targeting for non-prostate cancers was recently demonstrated in two distinct clinical trials (Morris et al., 2007, Clin. Cancer Res., 13:2707-13; Milowsky et al., 2007, J. Clin. Oncol., 25:540-547). The highly restricted presence of PSMA on prostate cancer cells and non-prostate solid tumor neovasculature makes it an attractive target for delivery of cytotoxic agents to most solid tumors.

Additional targeting agents are described in WO 2008/124632, which is incorporated herein by reference in its entirety. Other targeting moieties known or to be developed in the art are contemplated for use with the present disclosure.

### Methods of Making Delivery Compositions

Delivery compositions described herein can be prepared by any method known in the art, e.g., nanoprecipitation and emulsion methods. Additionally, microfluidics methods can be used to prepare the new delivery compositions.

Single-step nanoprecipitation methods are described in U.S. Pat. No. 5,118,528, which is incorporated herein by reference. These methods can be used to synthesize nanoparticles by mixing a solution containing a substance into another solution (i.e., a non-solvent) in which the substance has poor solubility. For example, polymeric (e.g., PLGA-PEG) nanoparticles can be made in which polymer solutions in either water-immiscible or water-miscible solvents are added to an aqueous fluid (i.e., the non-solvent). Such nanoprecipitation methods are also described, for example, in WO 2007/150030, which is incorporated herein by reference in its entirety. In one non-limiting example, a hydrophilic active agent conjugated to a biodegradable polymer having pendant functional groups is dissolved in a volatile, water-miscible organic solvent to form a first solution, and a hydrophobic active agent is dissolved in a volatile, water-miscible organic solvent to form a second solution. A third solution is prepared by dissolving a plurality of amphiphilic block copolymers in a water-miscible organic, and the first, second, and third solutions are combined such that nanoparticles having a hydrophobic polymeric core surrounded by the amphiphilic block copolymers are formed by precipitation.

Double emulsion methods of preparing particles are reviewed in Mundargi et al., 2008, Control. Release, 125:193-209, which is incorporated herein by reference in its entirety.

We have developed a microfluidic technology that enables preparation of PLGA-PEG NPs through rapid mixing, which allows for homogeneous conditions for nucleation and assembly of the nanoparticles (Karnik et al., 2008, Nano. Lett., 8:2906-12). Furthermore, by varying the flow rates of different polymeric precursors into the microfluidic device, the properties of the resulting nanoparticles can be systematically and reproducibly controlled. This technology can be employed for combinatorially mixing drug-functionalized polymers and ligand-functionalized polymers to generate a library of distinct targeted polymeric nanoparticles with varying biophysicochemical properties, each carrying two or more distinct active agents, e.g., anti-cancer drugs.

Nanoparticles can be prepared in a single step with distinct properties, starting from a well-defined batch of precursors (Anderson et al., 2004, Proc. Natl. Acad. Sci. USA, 101:16028-33). A single controlled nanoprecipitation step using microfluidic rapid mixing can provide reproducible self-assembly and remove variability due to dropwise mixing. All chemical conjugation steps can occur before formulation of the nanoparticles from the polymers, further minimizing variability. By varying the proportions of different precursors, nanoparticles with different sizes, charge, PEG coverage, and ligand density can be obtained. Microfluidic devices enable rapid mixing of nanoparticle precursor solutions into water, resulting in reproducible nanoprecipitation. This approach is robust and extremely simple in design, making it well-suited for preparing homogeneous nanoparticle formulations with distinct properties in an automated, high-throughput fashion. The ability to controllably and rapidly mix reagents and provide homogeneous reaction environments make microfluidic systems ideally suited for the synthesis of monodispersed nanoparticles (DeMello and DeMello, 2004, Lab on a Chip, 4:11 N-15N). The use of microfluidics can provide dramatic enhancement in the homogeneity of the resulting nanoparticles and a significant improvement in the reproducibility as compared to conventional nanoprecipitation without control over the mixing time. The methods yield nanoparticles with higher drug loading and slower drug release, possibly due to the formation of more compact nanoparticles with a more hydrophobic core. Furthermore, the simplicity of the method makes it amenable to automation, where input flow rates can be varied to control the composition and properties of the nanoparticles.

Other microfluidic systems for combinatorial semi-automatic nanoparticle synthesis can also be used for rapid synthesis of a library of nanoparticles with distinct biophysicochemical properties. One exemplary system consists of four computer-controlled syringe pumps, which can deliver different precursor polymers, drug, solvent, and water to a microfluidic device designed to intake four input streams of precursors (e.g., PLA-Drug A, PLA-Drug B, Drug C, and PLA-PEG-ligand). The precursors are first mixed in a certain ratio depending on the flow rates of each precursor, resulting in a distinct precursor combination. Following this mixing step, nanoparticles are synthesized from the given combination of precursors by nanoprecipitation using flow focusing to result in a nanoparticle formulation with characteristics determined by the unique combination of precursors.

For preparation of a library of particles with distinct formulations, after synthesis of one batch of nanoparticles, the flow rates are changed to result in another distinct formulation. The syringe pumps can be programmed to systematically vary the relative precursor flow rates to obtain a distinct nanoparticle formulation for each set of flow rates. After a brief period of time (e.g., 1-5 minutes) the flow rates are changed, resulting in the generation of new nanoparticle formulations serially at the rate of 10 to 30 distinct formulations per hour.

### Methods of Using Delivery Compositions

The invention further comprises preparations, formulations, kits, and the like, comprising any of the compositions as described herein. In some cases, treatment of a disease (e.g., cancer) cancer can involve the use of compositions or “agents” as described herein. That is, one aspect of the invention involves a series of compositions (e.g., pharmaceutical compositions) or agents useful for treatment of a disease (e.g., cancer or a tumor). These compositions can also be packaged in kits, optionally including instructions for use of the composition for the treatment of such conditions. These and other embodiments of the invention can also involve promotion of the treatment of a disease (e.g., cancer or tumor) according to any of the techniques and compositions and combinations of compositions described herein.

In some embodiments, compositions and methods of the invention can be used to prevent the growth of a tumor or cancer, and/or to prevent the metastasis of a tumor or cancer. In some embodiments, compositions of the invention can be used to shrink or destroy a cancer. It should be appreciated that compositions of the invention can be used alone or in combination with one or more additional anti-cancer agents or treatments (e.g., chemotherapeutic agents, targeted therapeutic agents, pseudo-targeted therapeutic agents, hormones, radiation, surgery, etc., or any combination of two or more thereof). In some embodiments, a composition of the invention can be administered to a patient who has undergone a treatment involving surgery, radiation, and/or chemotherapy. In certain embodiments, a composition of the invention can be administered chronically to prevent, or reduce the risk of, a cancer recurrence.

Compositions comprising particles of the present invention, in some embodiments, can be combined with pharmaceutically acceptable carriers to form a pharmaceutical composition, according to another aspect of the invention. As would be appreciated by one of skill in this art, the carriers can be chosen based on the route of administration as described below, the location of the target issue, the drug being delivered, the time course of delivery of the drug, etc.

A “pharmaceutical compositions” or “pharmaceutically acceptable” composition, as used herein, comprises a therapeutically effective amount of one or more of the compositions described herein, formulated together with one or more pharmaceutically acceptable carriers (additives) and/or diluents. As described in detail, the pharmaceutical compositions of the present invention can be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol (e.g., alpha-tocopheryl succinate), and the like; and metal chelating agents, such as citric acid, ethylenediamine tetraacetic acid (EDTA), sorbitol, tartaric acid, phosphoric acid, and the like.

The compositions of the present invention can be given in dosages, generally, at the maximum amount while avoiding or minimizing any potentially detrimental side effects. The compositions can be administered in effective amounts, alone or in a cocktail with other compounds, for example, other compounds that can be used to treat a disease (e.g., cancer). An effective amount is generally an amount sufficient to inhibit the disease (e.g., cancer) within the subject.

One of skill in the art can determine what an effective amount of the composition is by screening the composition using any of the assays described herein or other known assays. The effective amounts may depend, of course, on factors such as the severity of the condition being treated; individual patient parameters including age, physical condition, size, and weight; concurrent treatments; the frequency of treatment; or the mode of administration. These factors are well known to those of ordinary skill in the art and can be addressed with no more than routine experimentation. In some cases, a maximum dose be used, that is, the highest safe dose according to sound medical judgment.

Actual dosage levels of the active ingredients in the pharmaceutical compositions of this invention can be varied so as to obtain an amount of the active ingredient that is effective to achieve the desired therapeutic response for a particular patient, composition, and mode of administration, without being toxic to the patient.

The selected dosage level may depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and then gradually increasing the dosage until the desired effect is achieved.

In some embodiments, a compound or pharmaceutical composition of the invention is provided to a subject chronically. Chronic treatments include any form of repeated administration for an extended period of time, such as repeated administrations for one or more months, between a month and a year, one or more years, or longer. In many embodiments, a chronic treatment involves administering a compound or pharmaceutical composition of the invention repeatedly over the life of the subject. For example, chronic treatments can involve regular administrations, for example one or more times a day, one or more times a week, or one or more times a month. In general, a suitable dose such as a daily dose of a compound of the invention will be that amount of the compound that is the lowest dose effective to produce a therapeutic effect. Such an effective dose will generally depend upon the factors described above.

If desired, the effective daily dose of the active compound can be administered as two, three, four, five, six or more sub-doses administered separately at appropriate intervals throughout the day, optionally, in unit dosage forms.

While it is possible for a composition of the present invention to be administered alone, it can be administered as a pharmaceutical formulation (composition) as described above.

The compositions of the invention, in some embodiments, can be promoted for treatment of abnormal cell proliferation, diseases (e.g., cancers), or tumors, or includes instructions for treatment of accompany cell proliferation, cancers, or tumors, as mentioned above. In another aspect, the invention provides a method involving promoting the prevention or treatment of a disease (e.g., cancer) via administration of any one of the compositions of the present invention, and homologs, analogs, derivatives, enantiomers and functionally equivalent compositions thereof in which the composition is able to treat the disease. “Instructions” can define a component of promotion, and typically involve written instructions on or associated with packaging of compositions of the invention. Instructions also can include any oral or electronic instructions provided in any manner. The “kit” typically defines a package including any one or a combination of the compositions of the invention and the instructions, or homologs, analogs, derivatives, enantiomers and functionally equivalent compositions thereof, but can also include the composition of the invention and instructions of any form that are provided in connection with the composition in a manner such that a clinical professional will clearly recognize that the instructions are to be associated with the specific composition.

The kits described herein can also contain one or more containers, which can contain compounds such as the species, signaling entities, biomolecules and/or particles as described. The kits also can contain instructions for mixing, diluting, and/or administrating the compounds. The kits also can include other containers with one or more solvents, surfactants, preservatives, and/or diluents (e.g., normal saline (0.9% NaCl), or 5% dextrose) as well as containers for mixing, diluting or administering the components to the sample or to the patient in need of such treatment.

The compositions of the kit can be provided as any suitable form, for example, as liquid solutions or as dried powders. When the composition provided is a dry powder, the powder can be reconstituted by the addition of a suitable solvent, which can also be provided. In embodiments where liquid forms of the composition are sued, the liquid form can be concentrated or ready to use. The solvent can depend on the compound and the mode of use or administration. Suitable solvents for drug compositions are well known and are available in the literature.

The kit, in one set of embodiments, can comprise a carrier means being compartmentalized to receive in close confinement one or more container means such as vials, tubes, and the like, each of the container means comprising a specific composition. Additionally, the kit can include containers for other components, for example, buffers useful in the assay.

As used herein, a “subject” or a “patient” refers to any mammal (e.g., a human), such as a mammal that may be susceptible to a disease (e.g., cancer). Examples include a human, a non-human primate, a cow, a horse, a pig, a sheep, a goat, a dog, a cat, or a rodent such as a mouse, a rat, a hamster, or a guinea pig. A subject can be a subject diagnosed with the disease or otherwise known to have the disease (e.g., cancer). In some embodiments, a subject can be diagnosed as, or known to be, at risk of developing a disease. In certain embodiments, a subject can be selected for treatment on the basis of a known disease in the subject. In some embodiments, a subject can be selected for treatment on the basis of a suspected disease in the subject. In some embodiments, a disease can be diagnosed by detecting a mutation associate in a biological sample (e.g., urine, sputum, whole blood, serum, stool, etc., or any combination thereof. Accordingly, a compound or composition of the invention can be administered to a subject based, at least in part, on the fact that a mutation is detected in at least one sample (e.g., biopsy sample or any other biological sample) obtained from the subject. In some embodiments, a cancer can not have been detected or located in the subject, but the presence of a mutation associated with a cancer in at least one biological sample can be sufficient to prescribe or administer one or more compositions of the invention to the subject. In some embodiments, the composition can be administered to prevent the development of a disease such as cancer. However, in some embodiments, the presence of an existing disease can be suspected, but not yet identified, and a composition of the invention can be administered to prevent further growth or development of the disease.

It should be appreciated that any suitable technique can be used to identify or detect mutation and/or over-expression associated with a disease such as cancer. For example, nucleic acid detection techniques (e.g., sequencing, hybridization, etc.) or peptide detection techniques (e.g., sequencing, antibody-based detection, etc.) can be used. In some embodiments, other techniques can be used to detect or infer the presence of a cancer (e.g., histology, etc.). The presence of a cancer can be detected or inferred by detecting a mutation, over-expression, amplification, or any combination thereof at one or more other loci associated with a signaling pathway of a cancer.

### EXAMPLES

The invention is further described in the following examples, which do not limit the scope of the invention described in the claims.

### Example 1

### Synthesis of PLA-Pt

One strategy for the development of drug-functionalized polymers is based on the simple conversion of amino acids to their corresponding α-hydroxyl acids (i) (FIG. 11). First step involves the conversion of amine to hydroxyl via diazotization reaction using sodium nitrite in presence of an acid for 6 hours. This is a high yielding reaction where the resultant monomer was directly used for condensation polymerization in conjunction with lactic acid to give a polylactide copolymer, Poly-OBn. The condensation polymerization was performed using bulk reaction conditions at 150° C. for 3 hours with continuous argon purge, followed by further 3 hours under vacuum. The same polymer was made using ring opening polymerization (ROP) of the cyclic lactide monomer (ii) which was made via dehydration reaction of the α-hydroxyl acid under very dilute reaction conditions in toluene with 1% para-toluene sulfonic acid.

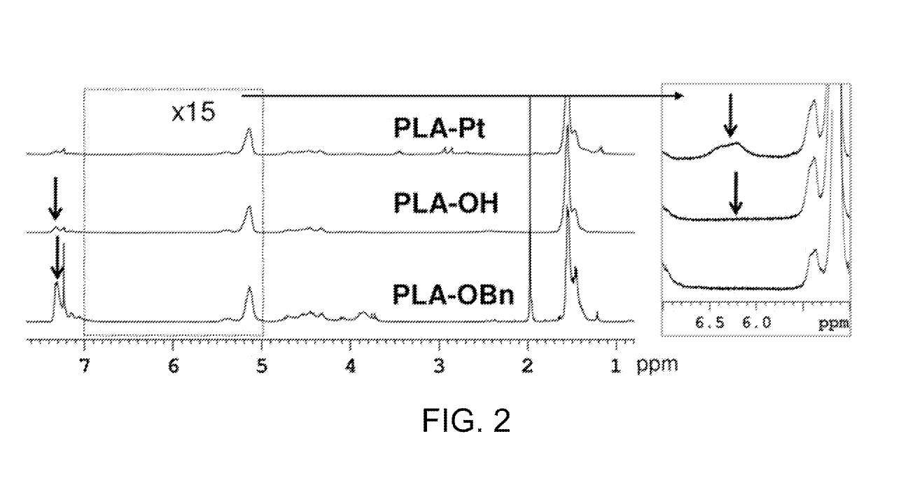

For the synthesis of high molecular weight polymers the ROP approach was more favorable. The benzyl protecting group prevents side reactions of the hydroxyl group during the polymerization. The hydroxyl functionalized biodegradable polylactide (PLA-OH) was then synthesized via benzyl deprotection using Pd/C catalyst at 50 psi pressure for 8 hours. Complete deprotection of benzyl groups was confirmed by ¹H-NMR spectroscopy by monitoring peak at 7.3 ppm (FIG. 2). For the synthesis of carboxyl functionalized polylactide, PLA-OH is treated with succinic anhydride or itaconic anhydride. As a demonstration for the development of biodegradable polymer with pendant hydrophilic drugs, a prodrug of cisplatin, platinum monosuccinate, was conjugated with the polymer using DCC/HOBT coupling to generate final PLA-Pt in DMF at room temperature after a 12-hour reaction (FIG. 12). The presence of platinum prodrug in PLA-Pt was visualized by ¹H-NMR spectroscopy as appearance of amine protons at 6.3 ppm after conjugation with prodrug (FIG. 2).

### Example 2

### Synthesis and Characterization of PLA-OH and PLA-Pt Nanoparticles

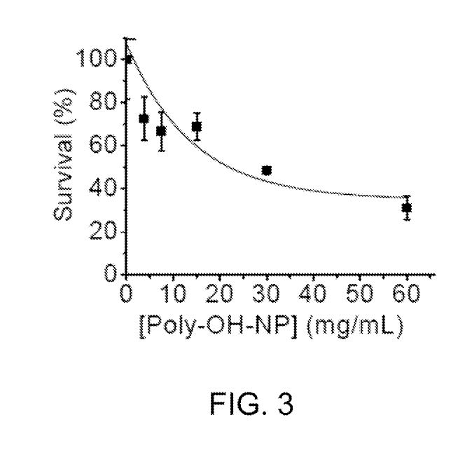

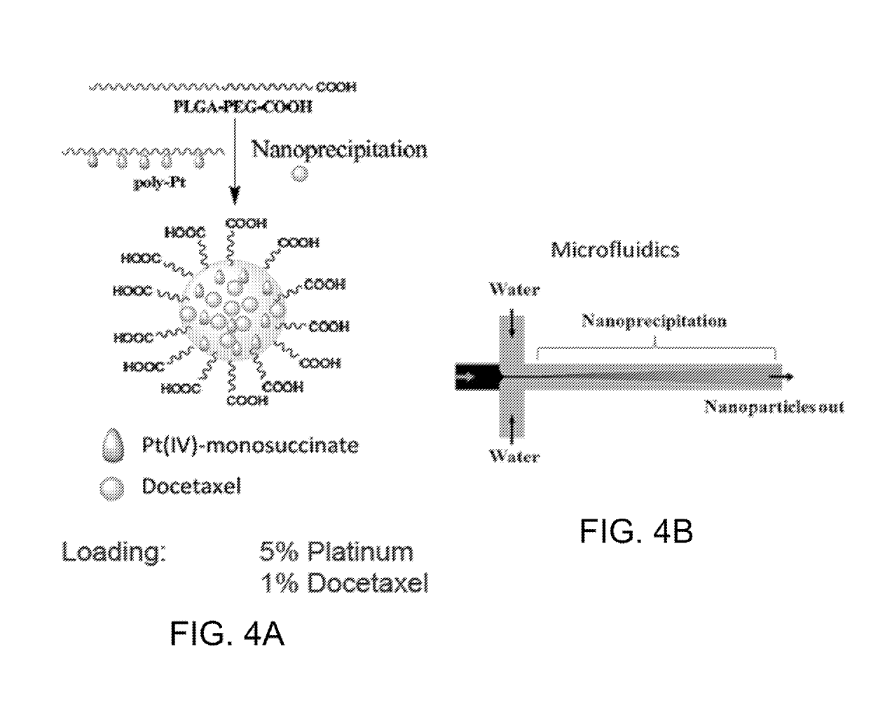

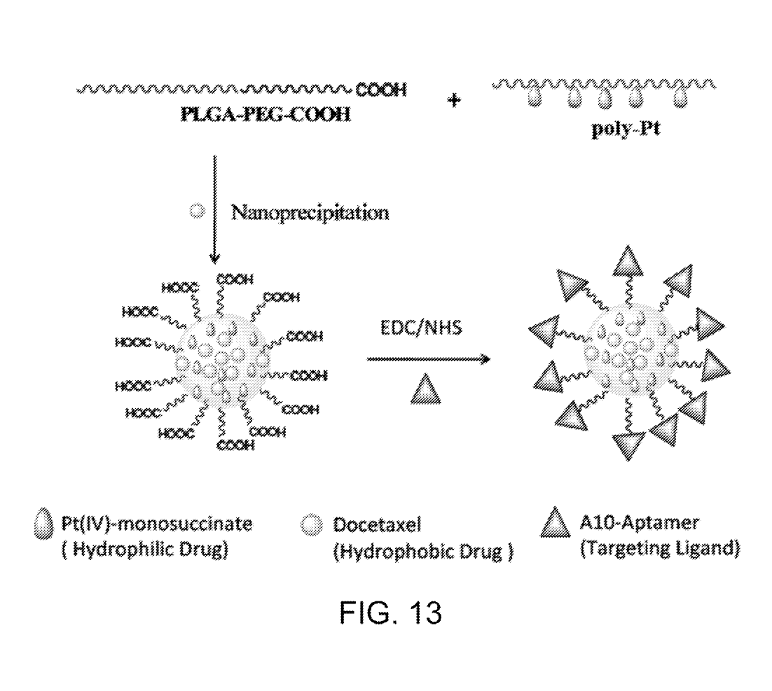

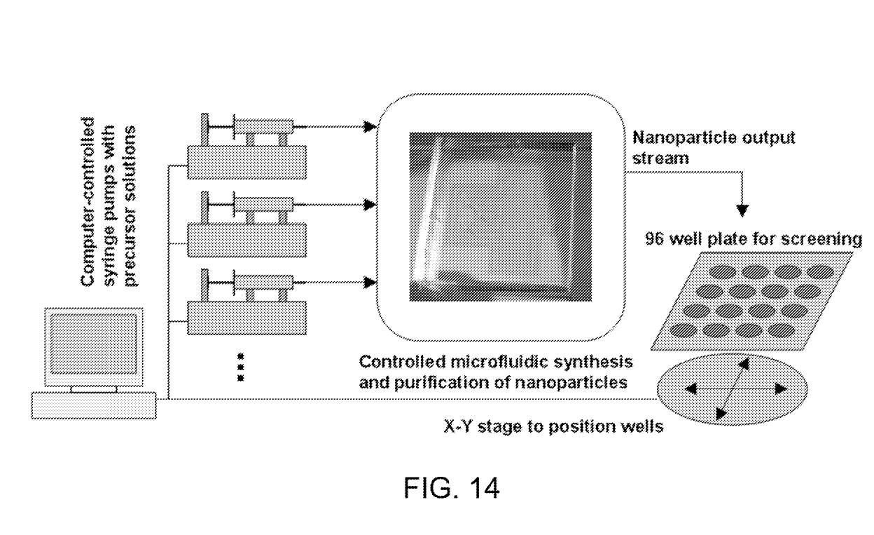

The in vitro toxicity of the PLA-OH polymer was tested by synthesizing nanoparticles with poly(lactic-co-glycolic acid)-polyethylene glycol (PLGA-PEG). The particles were relatively non toxic, with IC₅₀ values as high as 27 mg/mL (FIG. 3). Nanoparticles capable of delivering two drugs were made via nanoprecipitation using a micro-fluidics approach (FIG. 4B). In the present invention, we used standard PLGA-PEG with a functional group like carboxyl, along with the polylactide which contains hydrophilic drugs as pendants and was used for nanoprecipitation in presence of a hydrophobic drug. Once the nanoparticle was made, the particle was functionalized with a targeting ligand via coupling. A cisplatin prodrug was used as the hydrophilic drug, docetaxel as the hydrophobic drug, and an A10-Aptamer as a targeting ligand to target prostate cancer lines that overexpress prostate specific membrane antigen (PSMA) (FIG. 13).

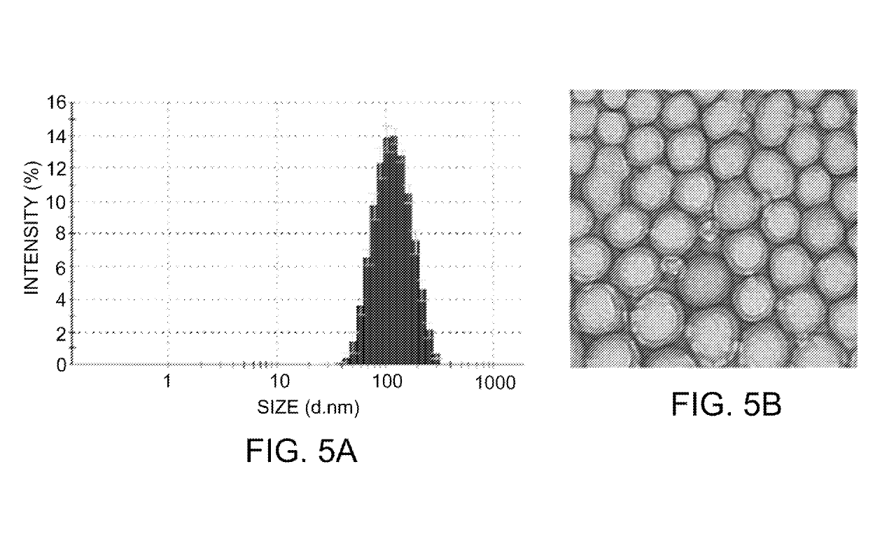

The presence of free PLGA typically increases the nanoparticle size during nanoprecipitaiton of PLGA-PEG. A microfluidics approach was used to achieve smaller size nanoparticles even in the presence of free PLGA (FIG. 5A, Table 1). The bulk nanoprecipitation yielded particles with sizes over 150 nm, while using the microfluidics approach we could get sizes around 100 nm. These particles were further characterized by transmission electron microscopy (TEM) (FIG. 5B). The TEM results confirmed the ˜100 nm size of these particles. The platinum and docetaxel loadings were 5% and 1% respectively in these particles. These values can be easily varied by varying the initial feed.

TABLE 1
Characterization of nanoparticles

| | Size (nm) | PDI |
|---|---|---|
| Poly-Pt-NP | 93 | 0.06 |
| Poly-Pt-Doce-NP | 89 | 0.08 |
| Doce-NP | 112 | 0.17 |
| NP | 109 | 0.13 |

### Example 3

### Electrochemistry of Poly-Pt

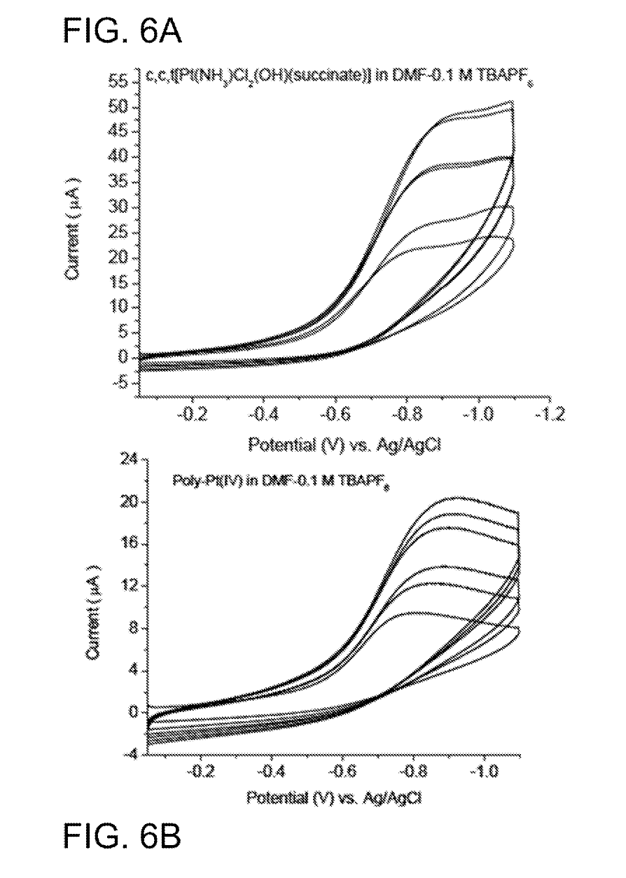

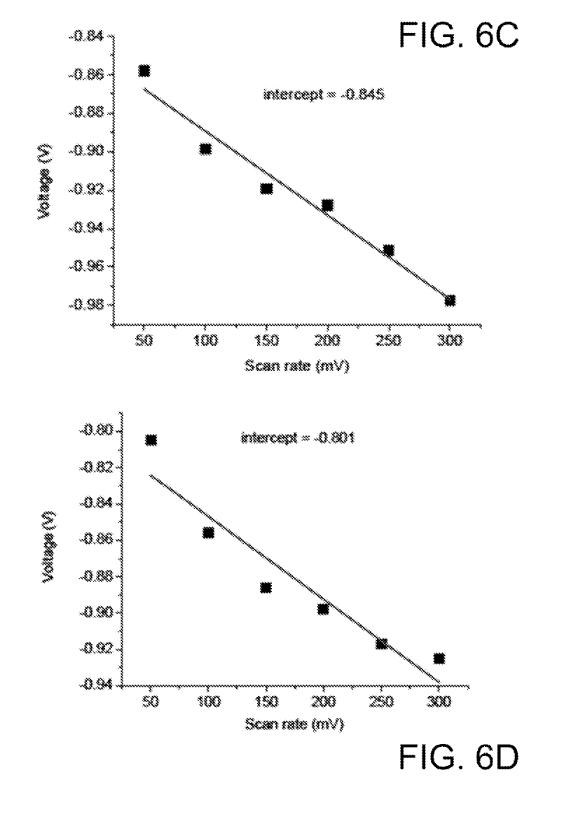

Electrochemical measurements were made at 25° C. on a EG&G PAR Model 263 Potentiostat/Galvanostat with electrochemical analysis software 270 and a three electrode set-up comprising a glassy carbon working electrode, platinum wire auxiliary electrode and a Ag/AgCl reference electrode. The electrochemical data were uncorrected for junction potentials. KCl was used as a supporting electrolyte. Poly-Pt is redox-active and displays irreversible cyclic voltammetric responses for the Pt(IV)/Pt(II) couple near −0.801 V vs Ag/AgCl at pH 7.4, and the value for the platinum monosuccinate prodrug was −0.850 vs. Ag/AgCl (FIGS. 6A-6D) under the same conditions. These reduction potentials suggest that presence of the polymeric backbone does not influence the electronic or steric environment of the platinum center and that this construct will effectively release the active dose of platinum to potentiate anticancer activity. The reduction potential value of poly-Pt indicates that it has the potential to avoid premature reduction in blood and will be reduced inside cells.

### Example 4

### In Vitro Release of Platinum and Docetaxel from the PolyPt-Doce-NPs

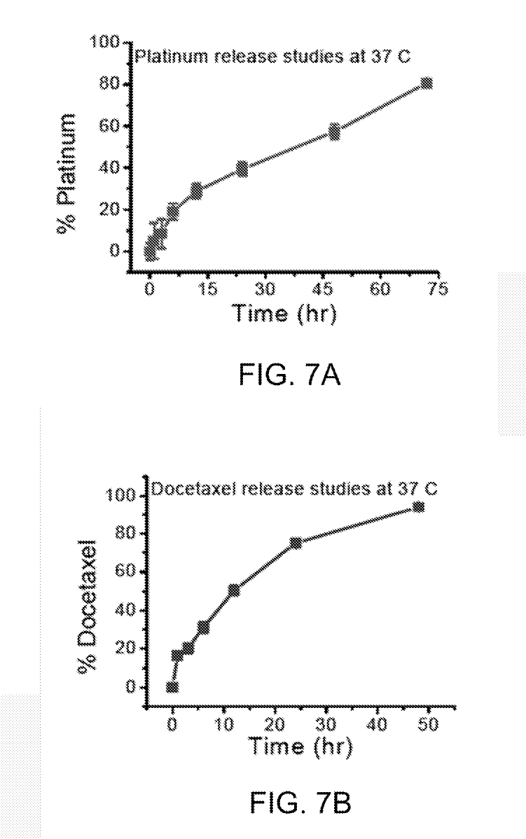

The controlled release kinetics of platinum(IV) and docetaxel from nanoparticles were studied. Controlled release of the drug candidates from the nanoparticles is an important advantage of the new particles, as the drugs become active only after they are released from the delivery vehicle. For the release study, we dialyzed the platinum and docetaxel-nanoparticles against 20 liters PBS of pH 7.4 at 37° C. to mimic the physiological conditions. The suspension of PolyPt-Doce-NPs in water were aliquoted (100 μL each) into several semipermeable minidialysis tubes (molecular weight cutoff 100 kDa, Pierce) and dialyzed against 20 liters of phosphate buffered saline (PBS) (pH 7.4) at 37° C. At a predetermined time, an aliquot of the nanoparticle suspension was removed and dissolved in acetonitrile. The platinum content released was determined by atomic absorption spectroscopy, and docetaxel release was quantified by HPLC using a standard calibration curve obtained with commercially available taxol. Controlled release of both platinum (FIG. 7A) and docetaxel (FIG. 7B) from these nanoparticles was observed. The results indicated that the system was able to release the drugs in a temporal fashion where docetaxel was released faster than the covalently linked platinum center. The timing of the release can be further extended by increasing the length of the polymer backbone.

### Example 5

### Cytotoxicity of Nanoparticles

The ability of the targeted drug releasing construct nanoparticles to promote cell death was determined using the MTT assay using human prostate cancer LNCaP and PC3 cells and compared against the standard compounds cisplatin and the Pt(IV)-monosuccinate prodrug. Human prostate cancer LNCaP and PC3 cells were obtained from the ATCC. LNCaP cells overexpress PSMA, whereas PC3 cells do not. The cells were incubated at 37° C. in 5% CO₂ in RPMI growth medium supplemented with 10% fetal bovine serum and 1% penicillin/streptomycin. The cells were passaged every 3 to 4 days and restarted from the frozen stock upon reaching passage number 20. Cytotoxic activity of the constructs was evaluated using the MTT assay. Solutions of the different constructs were freshly prepared in sterile PBS before use. All platinum constructs were quantified by atomic absorption spectroscopy. LNCaP and PC3 cells were seeded on a 96-well plate in 100 μL RPMI media and incubated for 24 hours.

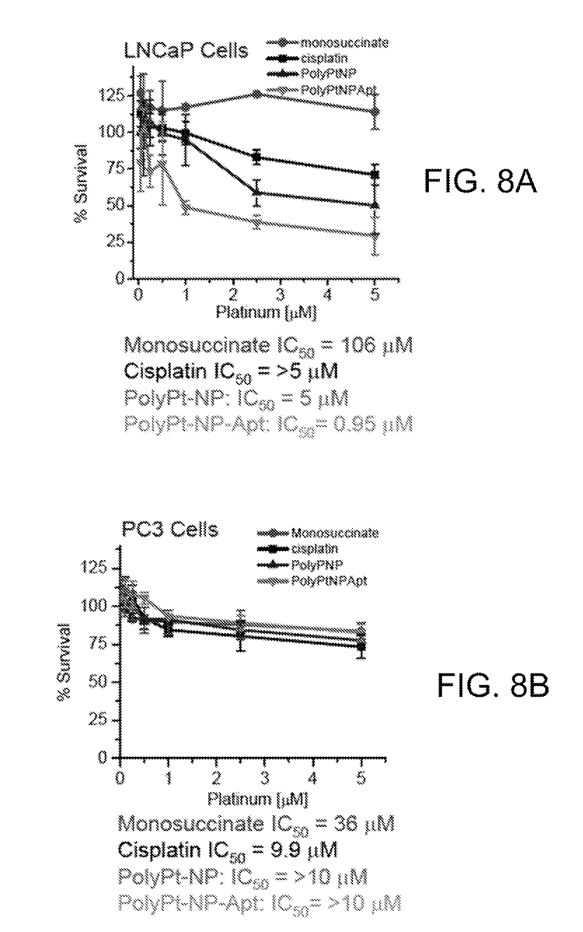

The cells were then treated with different constructs at varying concentrations and incubated for 12 hours at 37° C. Fresh medium was replaced after 12 hours incubation with nanoparticles, and the cells were incubated for a further 48 hours. The cells were then treated with 20 μL of 3-(4,5-dimethylthiazol-2-yl)-2,5-diphenyltetrazolium bromide (MTT) (5 mg/mL in PBS) and incubated for 5 hours. The medium was removed, the cells were lysed by adding 100 μL of DMSO, and the absorbance of the purple formazan was recorded at 550 nm using a microplate spectrophotometer. Each sample was assayed in triplicate in three independent experiments for each cell line. For each cell line, the Pt(IV)-monosuccinate prodrug (Monosuccinate) had the least effect on viability (FIGS. 8A-8B).

For PC3, the efficacies of the untargeted particles (PolyPt-NP) and targeted particles (PolyPt-NP-Apt) were approximately equivalent to that of unconjugated cisplatin (FIG. 8B). For the PSMA-overexpressing LNCaP cells, the efficacy of the untargeted particles (PolyPt-NP) was approximately equivalent to that of unconjugated cisplatin, whereas the efficacy of the targeted particles (PolyPt-NP-Apt) was approximately five-fold greater (FIG. 8A). Addition of the targeting aptamer also increased the efficacy in LNCaP cells of nanoparticles comprising docetaxel or both docetaxel and PolyPt (Table 2). This example demonstrates that the targeted nanoparticles and targeted dual-drug nanoparticles are effective at killing PSMA-expressing cancer cells.

TABLE 2
Comparison of IC₅₀ Values

| | IC₅₀ in | IC₅₀ in PC3 |
|---|---|---|
| | LNCaP cells (μM) | cells (μM) |
| Pt-Monosuccinate | 106 | 36 |
| Cisplatin | >5 | 9.9 |
| PolyPt-NP | 5 | >10 |
| PolyPt-NP-Apt | 0.95 | >10 |
| PolyPt-Doce-NP | 0.22 | 0.2 |
| PolyPt-Doce-NP-Apt | 0.09 | 0.36 |
| Doce-NP | 0.1 | 0.01 |
| Doce-NP-Apt | 0.02 | 0.01 |

[Pt] = 25 * [Doce]

### Example 6

### Targeted Endocytosis of PolyPt-NP-Apt

Polymeric nanoparticles can be taken up by cells through different processes, including phagocytosis and endocytosis. To study the internalization of PolyPt-NP-Apt, a green fluorescein-based cholesterol, 22-NBD-cholesterol, was included in the particles. Untargeted Poly-Pt-NP containing 22-NBD-cholesterol was used as a control. LNCaP cells were seeded on microscope coverslips (1.0 cm) at a confluence of 1×10⁵ cells per coverslip and grown overnight in a humidified incubator with 5% CO₂ at 37° C. in RPMI. The medium was changed, and a suspension of NBD-cholesterol-PolyPt-NP-Apt was added to a final fluorophore concentration of 10 μM. The cells were then incubated for 2.0 hours at 37° C. The medium was removed, and the cells were fixed using a 2% paraformaldehyde solution for 1 hour at room temperature. The cells were washed three times with PBS (pH 7.4). The cells were then permeabilized with 0.1% TRITON-X 100 in PBS for 10 minutes followed by five washes using PBS. The cells were then blocked with blocking buffer (PBS, 0.1% goat serum, 0.075% glycine) for 30 minutes at room temperature (RT). The cells were incubated for 1 hour at 37° C. with the early endosomal marker, mouse monoclonal EEA-1, in a wet box according to the manufacturer-recommended procedure. After two washes with PBS, the cells were blocked with blocking buffer for 30 minutes at RT and then incubated with the secondary Cy5 goat anti-mouse antibody for 1 hour at 37° C. After four washes with PBS and two washes with water, cells were mounted on microscope slides using the mounting solution [20 mM Tris (pH 8.0), 0.5% N-propyl gallate, and 70% glycerol] for imaging. Images were collected at 500 msec for both FITC and rhodamine channels.

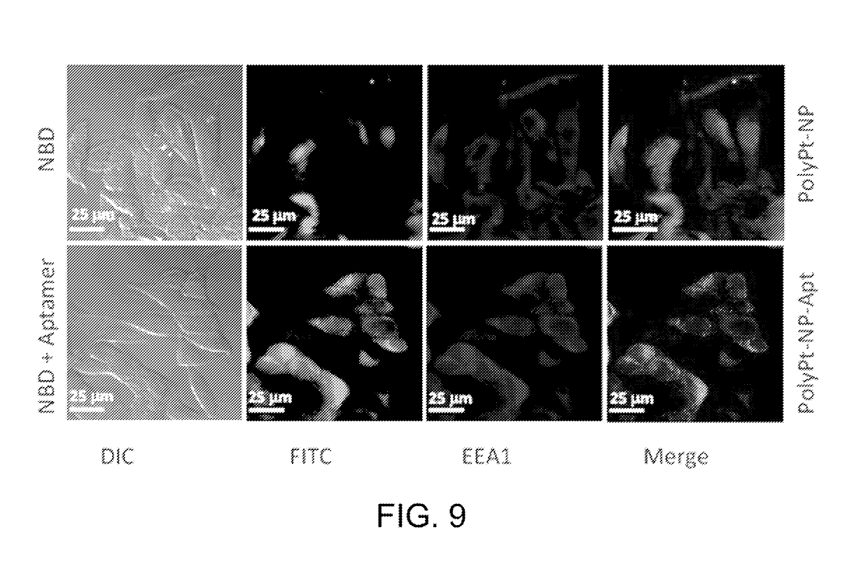

Clear evidence of targeted uptake of PolyPt-NP-Apt by PSMA overexpressing prostate cancer LNCaP cells via endocytosis was observed by using PolyPt-NP-Apt containing a green fluorescent labeled cholesterol derivative, 22-NBD-cholesterol (FIG. 9). Incubation of LNCaP cells with the cholesterol PolyPt-NP-Apt for 2 hours and use of the early endosomal marker EEA-1 antibody showed the complete internalization of these nanoparticles in the endosomes via aptamer targeted nanoparticle endocytosis (FIG. 9). By contrast, the non targeted Poly-Pt-NPs showed accumulation through out the cytoplasm (FIG. 9). This example demonstrates that targeted nanoparticles were taken up by endocytosis.

### Example 7

### Release of a Cytotoxic Dose of Cisplatin and the Formation of Pt-GG Adduct

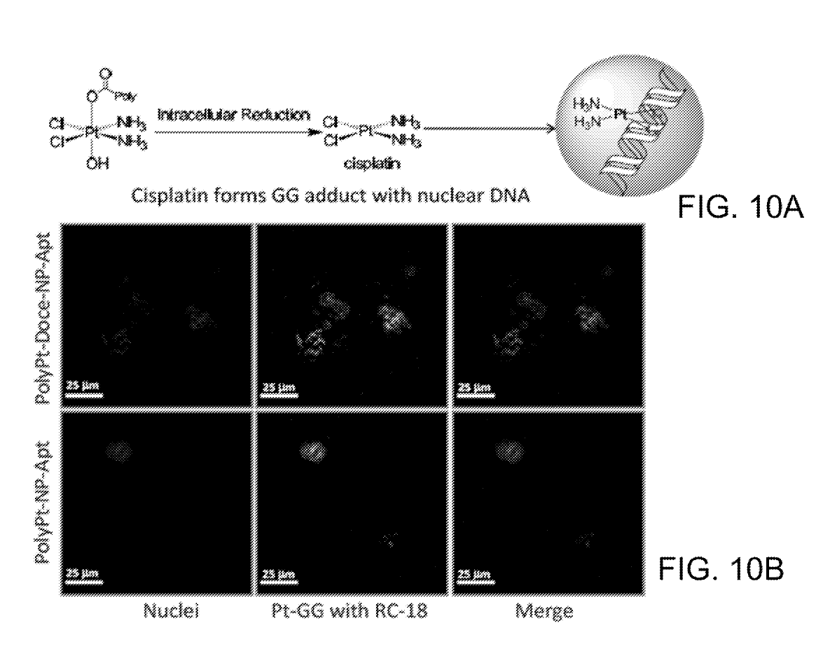

The anticancer activity of cisplatin is based on the formation of platination products in the nuclear DNA. Several of these adducts have been structurally identified, of which the guanine-guanine intrastrand cross-link cis-Pt(NH₃)₂d(GpG) (FIG. 10A), which represents >75% of total DNA platination. Detection of the platinum 1,2-d(GpG) adduct in cells was carried out by using an antibody specific to this adduct. Briefly, LNCaP cells were seeded in a six well plate using RPMI medium and incubated overnight at 37° C. Different constructs were added to a final concentration of 20 μM and incubated at 37° C. for 12 hours. The cells were then trypsinized, washed with PBS, resuspended in HAES-STERIL-PBS at a density of 1×10⁶ per mL and placed onto a pre-coated slide (ImmunoSelect, Squarix) to air-dry. Cell fixing was carried out at −20° C. in methanol for 45 minutes. Nuclear DNA was denatured by alkali (70 mM NaOH, 140 mM NaCl, 40% methanol v/v) treatment for 5 minutes at 0° C., and cellular proteins were removed by a proteolytic procedure involving two steps.

The cells were first digested with pepsin at 37° C. for 10 minutes and then with proteinase K at 37° C. for 5 minutes. After blocking with milk (1% in PBS, 30 minutes, 25° C.), the slides were incubated with anti-(Pt-DNA) Mabs (R-C18 0.1 mg/mL in milk) overnight at 4° C. After washing with PBS, immunostaining was performed by incubation with FITC-labeled goat anti-(rat Ig) antibody at 37° C. for 1 hour. The nuclei of the cells were stained by using Hoechst (H33258) (250 μg/L) and mounted using the mounting solution for imaging. We used a monoclonal antibody R-C18 specific for this adduct to learn whether cisplatin released from PolyPt-NP-Apt forms this adduct with nuclear DNA. After the 12-hour incubation of PSMA+LNCaP cells with PolyPt-NP-Apt, formation of 1,2-d(GpG) intrastrand cross-links was observed as antibody-derived green fluorescence in the nuclei of these cells (FIG. 10B). This example demonstrates that treatment of cells targeted nanoparticles of cisplatin prodrugs forms the expected adducts with nuclear DNA.

### Example 8

### Screening a Library of Targeted Nanoparticles

Using a microfluidic system a library of ˜500 drug containing PSMA-aptamer targeted nanoparticles is prepared and screened for desired characteristics. Nanoparticles are fabricated by the nanoprecipitation method that involves mixing of a water-miscible solution of precursors (co-block polymers, drug, etc.) into water. Precipitation of nanoparticles results since water is a poor solvent for the drug and polymers. Nanoparticle properties are controlled by (a) controlling the composition of the precursor solution, and (b) controlling mixing conditions such as mixing time, temperature, and flow ratio of water stream to precursor stream. These formulations are then tested for preferential uptake by PSMA-expressing prostate cancer cell line (LNCaP), versus PSMA-negative prostate cancer (PC3) and non-prostate (HeLa) cell lines using fluorescent dyes incorporated in the nanoparticles. This approach can provide optimized nanoparticles capable of evading macrophages after systemic administration while being able to get differentially taken up by prostate cancer cells.

By adding PEG to the surface of nanoparticles, the circulating half-life of the nanoparticles increases dramatically. The optimal physical and chemical properties of nanoparticles, including size of particles, surface modifications, surface charge, and ligand density, are identified that achieve minimal macrophage uptake and maximal specific drug delivery to cancer cells. The precursor solutions used are (a) PLGA-mPEG3400 and PLGA-PEG-COOH, for control of surface charge and hydrophobicity (b) PLGA for controlling size, (c) acetonitrile to control precursor concentrations, (d) drug/fluorescent reporter, (e) PLGA-PEG-ligand conjugate for targeting, (f) Drug-A functionalized PLGA and (g) Drug-B functionalized PLGA. Parameters such as precursor concentrations, mixing time, ratio of solvent to water, etc., are also varied in order to study the effect of these formulation parameters on nanoparticle size and zeta potential. The drug load and release kinetic of those formulations that demonstrate a favorable binding and uptake profile by PCa cells are further evaluated, and cell-based cytotoxicity is determined by MTT assays. At least 5 candidate formulations for each drug combination are identified and further tested for preferential PCa uptake.

### Example 9

### Evaluation of Delivery Compositions in an Animal Model

The delivery compositions described herein are evaluated in an animal model of prostate cancer. Severe combined Immunodeficient (SCID) mice (Taconic, Germantown, N.Y., USA) are injected subcutaneously with 1×10⁵ LNCaP cells mixed with 100 μA of Matrigel, into both rear hind limbs. After six days when the average tumor volume reaches around 150 mm³, mice are stratified into groups (five mice per group), so that the mean tumor volume in each group is comparable. At days 6, 9 and 14, animals are treated with PolyPt-Doce-NP, PolyPt-NP, Doce-NPs, Pt-Monosuccinate, Cisplatin (5 mg/kg), PolyPt-NP-Apt, PolyPt-Doce-NP-Apt, and Doce-NP-Apt via I.P. or i.v. injections. Tumor volume measurements begin on day 1 (tumor inoculation) and continue twice a week until the tumor volumes exceed 10% of the body size. The greatest effect on tumor growth is observed with the PolyPt-Doce-NP-Apt that contain both drugs and are targeted to PSMA on the LNCaP cells.

### Example 10

### In Vivo Characterization

Dose escalation studies are performed to determine the combination drug dose required for tumor reduction and/or tumor growth retardation in the xenograft and genetically engineered mouse models (GEMMs). MRI imaging of the mouse prostate is performed before and periodically after bioconjugate treatment for orthotopic tumor size comparison and efficacy measurement. Once an efficacious dose is determined, comparative efficacy studies are performed to compare drug containing nanoparticle-aptamer bioconjugates with similar nanoparticles lacking the aptamer targeting group; and with free drug and placebo in xenograft models and GEMMs. Biodistribution studies are also performed in tumor bearing mice using ¹¹¹In-labeled bioconjugates to evaluate the presence and relative concentration of bioconjugates in the tumor tissue.

Additionally, studies are performed to evaluate the concentration of released drugs in the tumor tissue, to determine the release kinetics. Bioconjugates generated from the PLA system are expected to have a slower release kinetic as compared to similar bioconjugates from the PLGA system. Polymer MW also alters the kinetic of drug release. Bioconjugates are developed that are capable of targeting PCa tissue specifically and result in efficacy against tumor growth.

In vivo acute dose toxicology screen of nanoparticle-aptamer bioconjugates: A cohort of 18 animals (6 groups of 3) is established to assay for the toxic effect of various concentration injections of bioconjugate polymers. A concentration less than LD50 is used in the subsequent biodistribution studies.

Biodistribution: For pharmacokinetic and biodistribution analysis non tumor BALB/c mice bearing animals are dosed with ¹¹¹In-labeled nanoparticle-aptamer bioconjugates or similar nanoparticles without the aptamer targeting group, through a lateral tail vein injection. The biodistribution is followed initially by SPECT-CT imaging (Gammamedica, X-SPECT) for 1-24 hours after injection, and the animals re sacrificed. Brain, heart, intestine, liver, spleen, kidney, muscle, bone, lung, lymph nodes, gut, and skin re excised, weighed, homogenized, and counted in a well counter (Wallace, Turku, Finland). Tissue concentration is expressed as percentage of injected dose per gram of tissue (% ID/g). Blood half-life is calculated from blood radioactivity at various time points after animals are injected (1, 2, 4, 8, 12, 18 and 24 hours).

Xenograft Tumor Induction: BALB/cnu/nu animals are anesthetized by avertin (225-240 mg/kg IP) and anesthesia is checked by applying pressure to the hind foot. Immediately prior to inoculation, the site of skin on the back is scrubbed with either povidine or chlorhexidine soap and wiped with 70% ethanol. The mouse is placed on its side on a clean surgical mat. The kidney is visible through the body wall and a flank incision is made along the long axis of kidney entering the subcutaneous area in the back. Through sharp and blunt dissection, the kidney is exteriorized and rested on the body wall. Approximately 0.5-1.0 mm³ tumor tissue freshly prepared from primary tumor specimens is implanted in the subrenal capsule through a small pocket created by a sharp nick followed by blunt dissection with fire rounded glass pipette. The kidney is eased back into the body cavity and skin is closed with wound clips. Tumors are expected to develop within 3-4 weeks and grow to a diameter (measured by MRI) of ˜5-10 mm. This size represents less than 1% of the mass of a typical 25-30 g mouse.

GEMMs of PCa: A genetically engineered mouse model of PCa has been established where the potent viral oncogene SV40 Tag was specifically expressed in epithelial cells of the prostate. Concomitant with the development of neoplasia, the expression of the hPSMA is triggered on the surface of tumor cells as seen in human tumors.

Dose escalating study: After tumors re established, an escalating dose of drug (two or three drug delivery system) nanoparticle-aptamer bioconjugates (0.5-5 mg/0.1 mL) is administered by lateral tail vein injection three times in 7 day intervals. Tumor volume is measured by MRI every 48 hours for 28 days.

Comparative efficacy study: After tumors are established, four different types of nanoparticles and two additional controls will be administered by lateral tail vein injection three times in 7 day intervals: 1) Drug nanoparticle-aptamer bioconjugates, 2) Placebo (dextran) nanoparticle-aptamer bioconjugates, 3) Drug nanoparticle without aptamers, 4) Placebo (dextran) nanoparticles without aptamers, 4) drug alone and 5) placebo alone. The animals are injected a fixed dose of compounds (0.5-5 mg/0.1 mL of particles as determined in dose escalating study, but not more than 50% of LD₅₀) resuspended in PBS or PBS alone through lateral tail vein. The animals are then allowed to recover in a heated recovery cage prior to analysis. Tumor volume is measured by MRI every 48 hours for 28 days.

### Example 11

### Combination Particles

To evaluate the synergistic effects of different metabolic activators on the action of platinum-based drugs, cisplatin and oxaliplatin, a number of combination were investigated by using the functionalized PLA derivatives (Table 3). We compared the effects of oxaliplatin and cisplatin when combined with lonidamine, dichloroacetate using in vitro studies.

TABLE 3
Combinations of platinum compounds with metabolic modulators

| Combination | Drug 1 | Drug 2 |
|---|---|---|
| Cisplatin- Lonidamine | Lonidamine 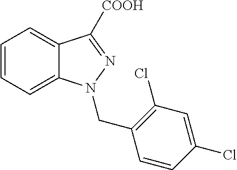 [SMILES: O=C(O)c1nn(Cc2ccc(Cl)cc2Cl)c2ccccc12] | Pt(IV)-hexanoate 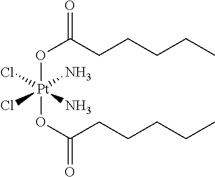 |
| Cisplatin-DCA | DCA 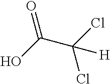 | Pt(IV)-hexanoate 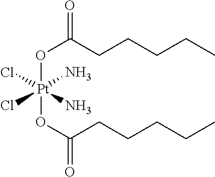 |
| Oxaliplatin- Irinotecan | Irinotecan 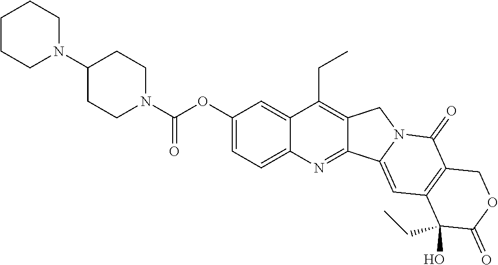 [SMILES: CCc1c2c(nc3ccc(OC(=O)N4CCC(N5CCCCC5)CC4)cc13)-c1cc3c(c(=O)n1C2)COC(=O)[C@]3(O)CC] | Oxaliplatin-prodrug 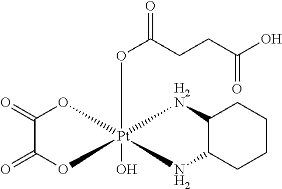 |

### Synthesis of PLA-COOH

A new lactide derivative with a —COOH functionality was synthesized by a reaction of PLA-OH and succinic anhydride (FIG. 15A). This new PLA derivative provided the opportunity to conjugate different drugs with —OH, —NH2 functionalities. The functionalized lactide PLA-COOH was characterized using 1H spectroscopy (FIG. 15B).

Cisplatin-Lonidamine Combination

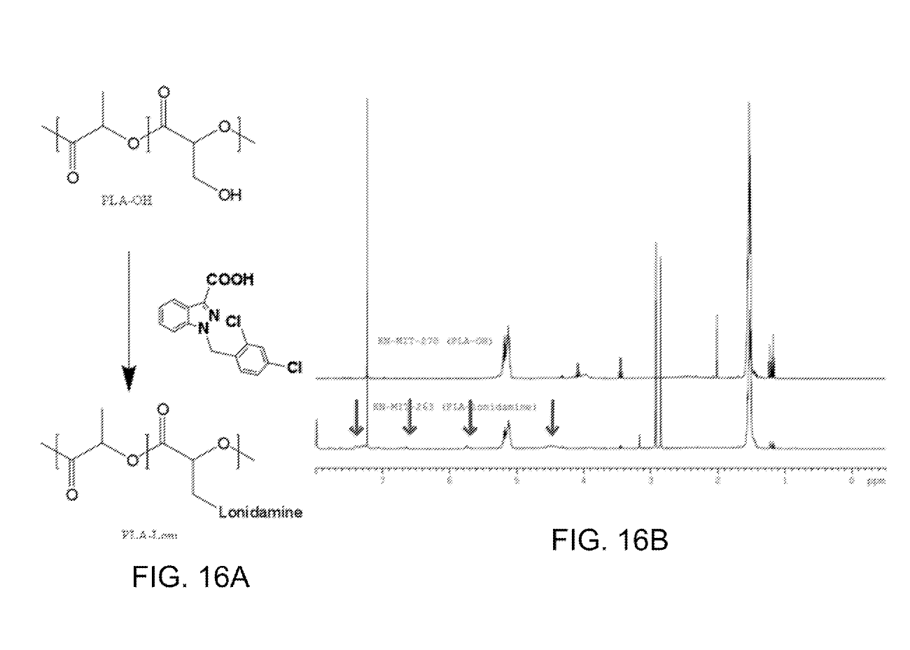

Lonidamine is a compound that inhibits mitochondrial hexokinase, selectively attacking the altered metabolism of cancerous cells. Studies have shown that it has synergistic effects in combination with several other chemotherapy drugs including cisplatin. We aimed to synthesize PLA-lonidamine (PLA-Loni) conjugate (FIG. 16A). Pt(IV)-hexanoate was encapsulated inside the PLA-lonidamine polymeric core by nanoprecipitating in the presence of PLGA-PEG-COOH. Lonidamine conjugated PLA was characterized by ¹H NMR (FIG. 16B).

Lonidamine and Pt(IV)-hexanoate co-encapsulated nanoparticles were synthesized using a nanoprecipitation method. Briefly, PLA-Loni, PLGA-PEG-COOH, and Pt(IV)-hexanoate were dissolved in acetonitrile and mixed together. This solution was slowly added to water and stirred at room temperature for 3 hours. These particles were purified by ultracentrifugation, and finally the particles were dispersed in deionized water. The size and polydispersity index of the particles were determined by using dynamic light scattering. Platinum and lonidamine encapsulation efficiencies and percent loadings were determined by using atomic absorption spectroscopy (AAS) for platinum and reverse-phase liquid chromatography for lonidamine.

In vitro cytotoxicity in prostate cancer cells of the dual-drug combination was evaluated using the MTT assay (Table 4). Combinations of lonidamine and Pt(IV) prodrug inside a nanoparticle gave enhanced cytotoxicity than the single agent inside a nanoparticle. For example, the toxicity of the dual drug combination in the prostate cancer LNCaP cells was increased by a factor of 5 with respect to Pt. The toxicity in PC3 cells was increased around 2 times with respect to Pt. The toxicity in DU145 cells was increased ˜2 times with respect to Pt. In all the three types of cell lines, the efficacy was increased over 1000 times with respect to lonidamine.

TABLE 4
Comparison of IC₅₀ values with various NPs and drugs against
LNCaP, PC3 and DU145 cells as determined by MTT assay

| | IC50 in LNCaP | | IC50 in PC3 | | IC50 in DU145 | |
|---|---|---|---|---|---|---|
| | cells (μM) | | cells (μM) | | cells (μM) | |
| | Pt | Loni | Pt | Loni | Pt | Loni |
| | conc. | conc. | conc. | conc. | conc. | conc. |
| HexaPt-NP | 0.5 | — | 1.4 | — | 0.64 | 00 |
| Loni-NP | — | >50 | — | >50 | — | >50 |
| PLA-Loni- | 0.103 | 0.556 | 0.85 | 22.1 | 0.31 | 2.1 |
| (HexaPt)-NP | | | | | | |

Cisplatin-DCA Combination

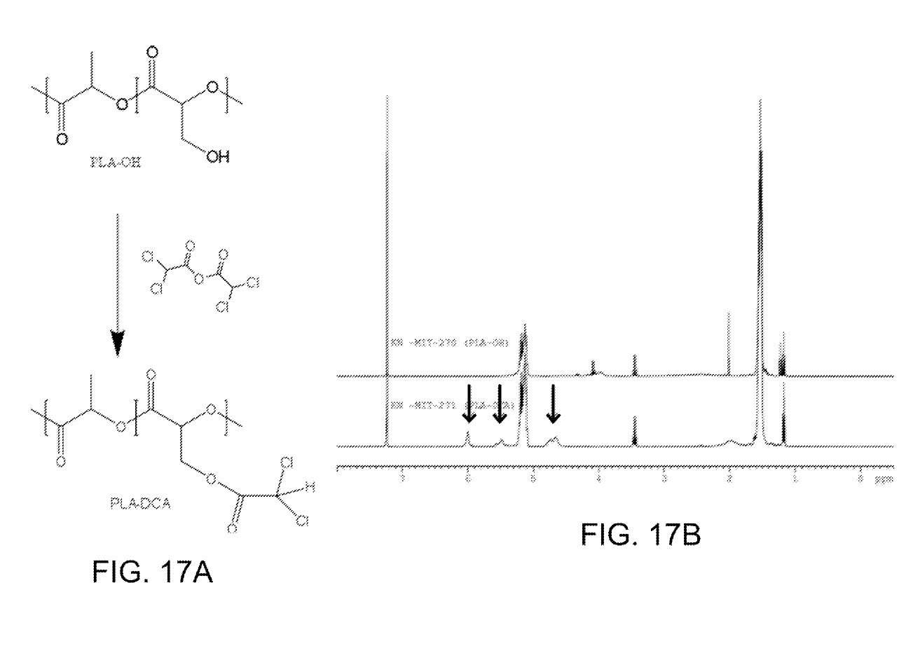

Dichloroacetate (DCA) is a small organic molecule that inhibits mitochondrial pyruvate dehydrogenase kinase (PDK), and thus promotes glucose oxidation over glycolysis. This phenomenon contributes to apoptosis of tumor cells. For a combination of cisplatin-DCA with high ratio of DCA, we synthesized PLA-DCA derivative (FIG. 17A). Coencapsulation of Pt(IV)-hexanoate in PLA-DCA in the presence of PLGA-PEG-COOH will result nanoparticles with both platinum and DCA. The PLA-DCA conjugate was characterized by 1H spectroscopy (FIG. 17B).

### Example 12

### Molecular Weights of Functional Polymers

In addition to above studies, various studies were carried out to vary the molecular weight of the functional polymer (PLA-OBn). Three different variables, dilution, catalyst concentration and initiator concentration were used for this purpose. The table below summarizes the results obtained from the study. From the results, the variation of catalyst concentration did the effect the resultant molecular weights significantly, but the variation of initiator concentration with respect to the monomers showed significant variations. In accordance to typical ring opening polymerization kinetics, by decreasing the concentration of initiator, the molecular weight of the resultant polymer increased (Table 5). On the other hand by varying the solvent of the polymerization the molecular weights were varied slightly, but did not follow any trend.

TABLE 5
Molecular weights of polymers

| | Initiator | | | PLA- | |
|---|---|---|---|---|---|
| Monomer:Functional | (Benzyl | Catalyst | Solvent | OBn | |
| Monomer | alcohol) | [Sn(Oct)2] | (toluene) | Mol. | |
| (Wt. ratio) | (wt. eqvi.) | (wt. eqvi.) | (wt. eqvi) | Wt. | PDI |
| 1:0.5 | 0.12 | 0.2 | 2 | 6100 | 1.4 |
| 1:0.5 | 0.09 | 0.2 | 2 | 6740 | 1.27 |
| 1:0.5 | 0.08 | 0.2 | 2 | 8950 | 1.3 |
| 1:0.5 | 0.12 | 0.2 | 2 | 6100 | 1.4 |
| 1:0.5 | 0.12 | 0.3 | 2 | 5900 | 1.41 |
| 1:0.5 | 0.12 | 0.2 | 2 | 6100 | 1.4 |
| 1:0.5 | 0.12 | 0.2 | 1.5 | 6560 | 1.33 |
| 1:0.5 | 0.12 | 0.2 | 1 | 5900 | 1.41 |

### Other Embodiments

A number of embodiments of the invention have been described. Nevertheless, it will be understood that various modifications may be made without departing from the spirit and scope of the invention. Accordingly, other embodiments are within the scope of the following claims.

1. A particle comprising a polymer matrix comprising a first active agent and a second active agent, wherein the polymer matrix is configured to provide release kinetics of each of the first and second active agents such that less than 50% of the each of the first and second active agents is released within the first two hours of suspending the particles in a neutral aqueous solution at about 37° C.

2. The particle of claim 1, wherein the polymer matrix further comprises one or more additional active agents.

3. A particle comprising a polymer matrix comprising a first active agent and a second active agent, wherein the polymer matrix is configured such that each of the first and second active agents has a half life in the circulation of a subject of at least two hours.

4. The particle of claim 3, wherein the polymer matrix further comprises one or more additional active agents.

5. The particle of claim 1, wherein the first active agent is conjugated to a biodegradable polymer.

6. The particle of claim 1, wherein the first active agent is conjugated to a biodegradable polymer having pendant functional groups.

7. The particle of claim 6, wherein the second active agent is conjugated to a biodegradable polymer.

8. The particle of claim 6, wherein the second active agent is conjugated to a biodegradable polymer having pendant functional groups.

9. A particle having multiple active agents, the particle comprising
(a) a hydrophobic polymeric core comprising
(i) a first active agent conjugated to a biodegradable polymer and;
(ii) a second active agent; and
(b) a hydrophilic layer that is surface-exposed.

10. The particle of claim 9, wherein the first active agent is conjugated to the biodegradable polymer through pendant functional groups on the polymer.

11. The particle of claim 9, wherein the biodegradable polymer is a block copolymer having a first end that is relatively hydrophobic and a second end that is relatively hydrophilic, wherein the hydrophobic core comprises the first end of the block copolymer conjugated to the first active agent, and wherein the hydrophilic layer comprises the second end of the block copolymer.

12. The particle of claim 9, wherein the hydrophilic layer comprises a plurality of amphiphilic block copolymers, each comprising a relatively hydrophobic end that interacts with the hydrophobic polymeric core and a relatively hydrophilic end that is surface-exposed.

13. The particle of claim 1, further comprising a targeting agent.

14. The particle of claim 1, wherein the first active agent and second active agent are independently selected from a biomolecule, bioactive agent, small molecule, drug, prodrug, drug derivative, protein, peptide, vaccine, adjuvant, fluorescent molecule, or polynucleotide.

15. The particle of claim 1, wherein the first and second active agents are, respectively, paclitaxel or docetaxel and gefitinib; gefitinib and paclitaxel or docetaxel; oxaliplatin (or oxaliplatin prodrug) and irinotecan; irinotecan and oxaliplatin (or oxaliplatin prodrug); paclitaxel and tubacin; tubacin and placlitaxel; lonidamine, dichloroacetate, alpha-tocopheryl succinate, betulinic acid, or resveratrol and Pt(IV) hexanoate; Pt(IV) hexanoate and lonidamine, dichloroacetate, alpha-tocopheryl succinate, betulinic acid, or resveratrol; alpha-tocopheryl succinate or methyl jasmonate and docetaxel; or docetaxel and alpha-tocopheryl succinate or methyl jasmonate.

16. A method of formulating a particle comprising at least two active agents, the method comprising:
providing a first and second active agent;
conjugating the first active agent, or a prodrug or derivative thereof, to a biodegradable polymer having pendant functional groups; and
preparing a particle comprising the conjugated first active agent and the second active agent.

17. The method of claim 16, further comprising conjugating the second active agent, or a prodrug or derivative thereof, to a biodegradable polymer having pendant functional groups.

18. The method of claim 16, wherein conjugating the first active agent and/or the second active agent to a biodegradable polymer having pendant functional groups imparts compatibility of the first and second active agents for formation of a particle.

19. A method of tempospatially controlling administration of two or more active agents to a subject, the method comprising:
providing a particle comprising a polymer matrix comprising a first active agent and a second active agent, wherein the polymer matrix is configured to provide desired tempospatial release kinetics of each of the first and second active agents; and
administering the particle to a subject such that the first and second active agents are released from the particle with the desired tempospatial release kinetics.

20. A pharmaceutical composition for intravenous, intra-arterial, oral, transdermal, transmucosal, intraperitoneal, intracranial, intraocular, epidural, intrathecal, topical, enema, injection, pulmonary route or infusion delivery comprising a plurality of particles of claim 1.
